(19) United States
(12) Patent Application Publication
(10) Pub. No.: US 2025/0311960 A1
(43) Pub. Date: Oct. 9, 2025

(54) DEVICES, SYSTEMS, AND METHODS FOR COGNITIVE VULNERABILITY ASSESSMENT AND TREATMENTS THEREOF

(71) Applicant: Duke University, Durham, NC (US)

(72) Inventors: Leah C. Acker, Durham, NC (US); Marty G. Woldorff, Durham, NC (US)

(21) Appl. No.: 19/169,408

(22) Filed: Apr. 3, 2025

Related U.S. Application Data
(60) Provisional application No. 63/574,463, filed on Apr. 4, 2024.

(51) Int. Cl. A61B 5/374; A61B 5/00; A61B 5/16; A61B 5/291

(52) U.S. Cl. CPC A61B 5/374; A61B 5/168; A61B 5/291; A61B 5/4088; A61B 5/4836

(57) ABSTRACT

Provided herein is a system for detecting neurocognitive weakness in a subject. The system can include one or more electrodes and at least one processor. The one or more electrodes can be operable to measure a first set of one or more EEG signals of the subject and a second set of one or more EEG signals of the subject. The at least one processor can be configured to receive the first set and the second set, determine a first alpha power from the first set and a second alpha power from the second set, determine a difference between the first alpha power and the second alpha power, and generate an alpha reactivity based on the difference. The first set can be measured when the subject is in an eyes-closed state. The second set can be measured when the subject is in an eyes-open state.

### CROSS-REFERENCE TO RELATED APPLICATIONS

This application claims priority to U.S. Provisional Application No. 63/574,463 filed Apr. 4, 2024, the contents of which are entirely incorporated by reference herein.

### STATEMENT REGARDING FEDERALLY SPONSORED RESEARCH OR DEVELOPMENT

This invention was made with government support under Federal Grant Nos. UH2 AG056925, P30 AG028716, R03 AG078891, and T32 GM008600 awarded by the National Institutes of Health. The federal government has certain rights to this invention.

### FIELD OF DISCLOSURE

The present disclosure relates to systems and methods for detecting and treating neurocognitive weakness and/or predicting neurocognitive weakness.

### BACKGROUND

Postoperative delirium (POD) is a syndrome of acute fluctuating changes in attention and consciousness that affects up to 50% of surgery patients 65 and older, increases the risk for Alzheimer's disease (AD) and AD-related dementias (ADRD), and accelerates dementia progression. Yet, interventions for POD, as well as other types of cognitive impairments, are limited because its pathophysiologic mechanisms are poorly understood.

Therefore, there is a need for systems and methods to predict and treat POD and other deficiencies of neurocognitive processes.

### SUMMARY

Provided herein is a system for detecting neurocognitive weakness in a subject. The system can include one or more electrodes and at least one processor. The one or more electrodes can be operable to measure a first set of one or more EEG signals of the subject and a second set of one or more EEG signals of the subject. The at least one processor can be configured to receive the first set of one or more EEG signals and the second set of one or more EEG signals, determine a first alpha power for the first set of one or more EEG signals and a second alpha power for the second set of one or more EEG signals, determine a difference between the first alpha power and the second alpha power, and generate an alpha reactivity defined by the difference. In some aspects, the first set of one or more EEG signals can be measured when the subject is in an eyes closed state. In some aspects, the second set of EEG signals can be measured when the subject is in an eyes open state.

In some aspects, the at least one processor can be further configured to compare the alpha reactivity to a threshold or degree of alpha reactivity. In some aspects, the at least one processor can be further configured to determine, based on the comparison of the alpha reactivity to the threshold or the degree of alpha reactivity, an attentiveness of the subject. In some aspects, the attentiveness of the subject can include one of attentive, likely to become inattentive, moderately inattentive, and chronically inattentive.

In some aspects, the one or more electrodes can include at least two frontal electrodes. In some aspects, the at least two frontal electrodes can be operable to contact a forehead of the subject. In some aspects, the one or more electrodes can further include at least one occipital electrode and at least one frontal electrode. In some aspects, the at least one occipital electrode can be operable to contact a scalp of the subject over an occipital lobe of the subject.

In some aspects, the at least one processor can further be configured to separate the first alpha power and the second alpha power into one or more sub-bands. In some aspects, the one or more sub-bands can include a low-frequency sub-band and a high-frequency sub-band. In some aspects, the at least one processor can be further configured to determine a difference between the first alpha power and the second alpha power in the one or more sub-bands, generate one or more sub-band alpha reactivities for each of the one or more sub-bands, and determine one or more conditions of the subject based on the one or more sub-band alpha reactivities.

In some aspects, the at least one processor can be further operable to determine a treatment for the subject based on the alpha reactivity. In some aspects, the system can further include a support structure. In some aspects, the one or more electrodes are coupled to the support structure.

Further provided herein is a method for detecting and treating neurocognitive weakness in a subject. The method can include measuring, via one or more electrodes in contact with the subject, a first set of one or more EEG signals of the subject and a second set of one or more EEG signals of the subject, sending the first set of one or more EEG signals and the second set of one or more EEG signals to at least one processor, determining, via the at least one processor, a first alpha power for the first set of one or more EEG signals and a second alpha power for the second set of one or more EEG signals, determining a difference between the first alpha power and the second alpha power, generating an alpha reactivity defined by the difference, comparing the alpha reactivity to one or more alpha reactivity thresholds or degrees, determining a treatment based on the comparison of the alpha reactivity to the one or more alpha reactivity thresholds or degrees, and administering the treatment to the subject. The first set of one or more EEG signals can be measured when the subject is in an eyes closed state. The second set of one or more EEG signals can be measured when the subject is in an eyes open state.

In some aspects, the method can further include determining, based on the comparison of the alpha reactivity to the one or more alpha reactivity thresholds or degrees, a robustness of an attentional or cognitive control system of the subject. In some aspects, the robustness of the attentional or cognitive control system can include one of attentive, likely to become inattentive, moderately inattentive, and chronically inattentive.

In some aspects, the method can further include separating the first alpha power and the second alpha power into one or more sub-bands. In some aspects, the method can further include determining a difference between the first alpha power and the second alpha power for each of the one or more sub-bands, generating one or more alpha sub-band reactivities for each of the one or more sub-bands, and determining one or more conditions of the subject based on the one or more alpha sub-band reactivities.

In some aspects, the one or more conditions can include one or more of inattentiveness, post-operative delirium, MCI, dementia, depression, anxiety, ADHD, and other neurological conditions. In some aspects, the one or more sub-bands can include a lower frequency sub-band and a high frequency sub-band. In some aspects, the treatment can include one or more of neurofeedback or biofeedback therapy, family counseling, administration of medications, and/or adjustment of anesthesia-inducing procedures and/or drugs.

Other aspects and iterations of the invention are described more thoroughly below.

### BRIEF DESCRIPTION OF FIGURES

The patent or application file contains at least one drawing executed in color. Copies of this patent or patent application publication with color drawing(s) will be provided by the Office upon request and payment of the necessary fee.

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 illustrates a system for detecting neurocognitive weakness in using measures of electroencephalogram (EEG) recordings of brain activity.

FIG. 2 illustrates a degree scale of reactivity of EEG power in the alpha frequency band (˜7-13 Hz), with reactivity defined as the alpha power in one condition (e.g., eyes closed) vs. another condition (eyes-open).

FIG. 3 illustrates an EEG measurement device in one example using a scalp band with several recording electrodes.

FIG. 4 illustrates an example of the montage (placement) of EEG electrodes on the scalp in a full scalp coverage.

FIG. 5 is a flowchart of a method for detecting neurocognitive weakness in one example.

FIG. 6 is a diagram illustrating an example of a computing system operable to record and/or analyze the EEG signals.

FIG. 7A is a series of bar graphs illustrating an experimentally determined association between EEG alpha reactivity and attentional impairment severity.

FIG. 7B is a series of EEG power graphs illustrating an association between EEG alpha reactivity and delirium severity.

FIG. 8A illustrates whole-head preoperative time-frequency plots of EEG spectral power in the eyes-open condition, eyes-closed condition, and the difference between them (eyes closed minus eyes open), showing alpha attenuation (reactivity) in the difference, for subjects who were attentive after surgery (n=31) compared with those who were inattentive (n=40, left column, top row), the subgroup of inattentive patients who were newly inattentive (n=22, middle column, top row), and the chronically inattentive subgroup (n=18, right column, top row). Shading reflects the 95% confidence interval (CI) around the mean at each frequency for each group. The alpha band (7-13 Hz) is denoted with vertical dashed lines. Note the substantial difference in attenuation of power in the alpha band between the attentive and inattentive groups. Power spectral densities for eyes closed power considered alone (middle row) and eyes open power considered alone (bottom row) are shown in the same manner. *P<0.05 for age- and Mini-Mental Status Examination (MMSE)-adjusted logistic regression for alpha attenuation vs attention status (attentive vs inattentive) with multiple-comparison adjustment. **P<0.05 across the data shown in the graphs in both the middle and right column for a given row using the age- and MMSE-adjusted proportional odds regression for greater inattention chronicity.

FIG. 8B illustrates whole-head preoperative alpha power attenuation for the full alpha band (left bar group), lower alpha band (middle bar group), and higher alpha band (right bar group). As per multivariate, multiple-comparison-corrected analyses, the full alpha band and the low alpha sub-band showed a significant gradated response between attentive, newly inattentive, and chronically inattentive subjects (P<0.05, denoted by asterisks), whereas in the high alpha sub-band attenuation was not significantly gradated across the three inattention chronicity conditions. Error bars reflect the 95% CI around the mean.

FIG. 8C illustrates box plots of the whole-head alpha attenuation with median line (vertical middle), 25-75th percentile markers (box limits), full range and outliners denoted for distinct levels of delirium severity from 0 (least severe, brown) to ≥3 (most severe, blue), presented for visualization purposes. Alpha attenuation was greatest for subjects with the lowest delirium severity score (brown box).

FIG. 9A illustrates topographic plots showing the scalp distribution of alpha attenuation (in dB) with eyes opening (eyes-closed vs. eyes-open) for attentive (left), inattentive (left middle), newly inattentive (right middle), and chronically inattentive (right) subjects for the full alpha band (top row), lower alpha band (middle row), and higher alpha band (bottom row).

FIG. 9B shows the full alpha band (7-13 Hz) attenuation over frontal scalp locations, shown via horizontal box plots, with the vertical median line, 25-75th percentile denoted via box, and full range and outliers shown.

FIG. 10 illustrates a clinical STROBE diagram for a study in one example.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “substantially” is defined to be essentially conforming to the particular dimension, shape or other word that substantially modifies, such that the component need not be exact.

The terms “comprising,” “including” and “having” are used interchangeably in this disclosure. The terms “comprising,” “including” and “having” mean to include, but not necessarily be limited to the things so described.

The term “coupled” as used herein is defined as connected, whether directly or indirectly through intervening components, and is not necessarily limited to physical connections. The connection can be such that the objects are permanently connected or releasably connected, either physically or functionally.

The term “attentive” as used herein is defined as a neurological status of a subject. An attentive subject is a subject who passes or would pass an attentional neurological examination such as Confusion Assessment Method-defined delirium (3D-CAM), a Mini-Mental Status Examination (MMSE), or other attention related neurological examination or who would be deemed attentive based on clinical judgment.

The term “inattentive” as used herein is defined as a neurological status of a subject. An inattentive subject is a subject who fails or would fail an attentional neurological examination such as Confusion Assessment Method-defined delirium (3D-CAM), a Mini-Mental Status Examination (MMSE), or other attention related neurological examination or who would be deemed as such based on clinical judgment.

Currently, attentiveness of a subject is determined based on examinations, such as a Mini-Mental Status Examination. These examinations provide physicians with information regarding the subject's neurocognitive function. Physicians may also use alternative measures, clinical experience and clinical judgment to assess attentiveness. However, many subjects who pass an examination may still be at risk of developing neurocognitive weakness (e.g., inattentiveness) when exposed to a neurologically impactful event (e.g., administration of anesthesia). It was discovered that alpha reactivity (a difference in alpha power in EEG signals between an eyes-closed and eyes-open state) can be used to determine whether a subject is attentive or inattentive. Further, it was surprisingly found that alpha reactivity (the difference in EEG alpha power between an eyes-closed and eyes-open state) can be a valuable biomarker predictor for when a subject is likely to become inattentive or have deficiency in attentiveness after a neurologically impactful event (e.g., surgery or administration of anesthesia). The systems and methods described herein can, based on measured and calculated alpha reactivity, serve to determine whether a patient has a robust attentional control system, or is likely to have attentional deficiencies or to show them after a neurologically impactful event. Alpha reactivity can therefore be an invaluable measure for determining and predicting neurocognitive weakness.

Decreased neurocognitive function may not be apparent in adults until after a stressor such as illness, injury, surgery, anesthesia, and the like. Yet, patients who display cognitive weakness after a stressor likely had brain vulnerability before the stressor. For example, brain electrical activity captured by electroencephalography (EEG) recordings may show a tendency toward POD sub-features, such as inattention. Decreased preoperative oscillatory EEG alpha power when going from the eyes closed to eyes open condition is associated with inattention after surgery, even after controlling for age and baseline cognition. This relationship remains when considering only the frontal electrodes.

The systems and methods described herein are operable to detect and/or predict neurocognitive weakness in the attentional/cognitive control system. In some examples, the systems and methods can detect cognitive dysfunctions or weakness after a neural system stressor and/or predict that cognitive dysfunction is likely to occur. The systems and methods can be used as a routine test to monitor changes in cognitive function. The systems and methods can provide one or more cognitive control manipulations and measuring the subject's brain's reaction to the manipulation(s). The EEG alpha power of the brain is recorded during the manipulation and then compared to threshold or degree prediction values. In an example, the attenuation of EEG power in the alpha frequency band (7-13 Hz) is measured.

In some examples, the systems and methods use attenuation of EEG alpha power in individuals who appear cognitively normal prior to surgery to predict the likelihood of post-operative attentional deficits after surgery. Further, abnormal EEG alpha power modulation can be used to predict persistence or worsening of pre-existing cognitive impairments after surgery or other major physiological stressors. Because frontal-only EEG measurements can be collected easily in a few minutes preoperatively, the disclosed EEG-based systems and methods that use only frontal EEG channels can be incorporated into routine preoperative evaluations for older adults.

In some examples, the cognitive control manipulation comprises an eye opening and closing exercise, although it is to be understood that the manipulation can include other types of manipulations. Alpha power dominates the EEG in the inwardly focused, eyes-closed state, because alpha power inversely correlates with both externally directed attention and global arousal. In contrast, when the subject opens their eyes, visual input (or the brain's attentional anticipation of this input) activates a system of thalamic nuclei and cortical structures to induce widespread cortico-cortical and corticothalamic interactions. Thus, these interactions when the eyes are opened desynchronize EEG alpha oscillations and reduce EEG alpha power, a phenomenon known as alpha reactivity. The present disclosure proposes that this alpha reactivity reflects the integrity of specific neural circuits and structures associated with neurocognitive resilience. Additionally, an impaired neurocognitive resilience may affect the autonomic nervous system, the hypothalamic-pituitary axis, and/or cholinergic or dopaminergic control structures.

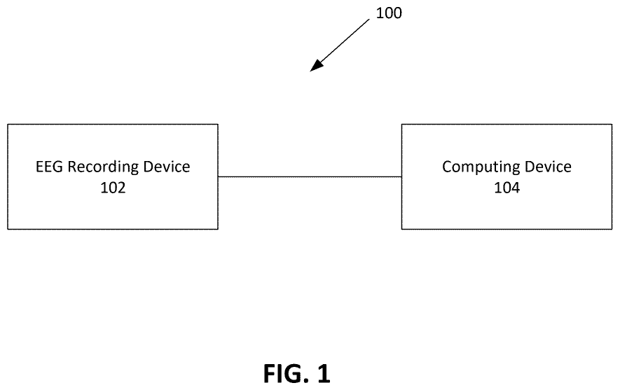

FIG. 1 illustrates a system 100 for detecting neurocognitive weakness of a subject and/or predicting future neurocognitive weakness of a subject. The system 100 can include an EEG recording device 102 and a computing device 104. The EEG recording device 102 can include any suitable channels for measuring EEG activity. In some examples, the EEG recording device 102 can include one or more electrodes operable to measure EEG signals of the subject. The computing device 104 can be operable to analyze the EEG signals and determine the power in useful frequency bands (such as alpha) from the EEG signals as described herein.

In some examples, the EEG recording device 102 can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or more electrodes.

In some examples, the EEG recording device 102 can include one or more frontal electrodes. The frontal electrodes can be operable to record EEG signals arising primarily from the frontal lobe of the subject. In some examples, the EEG recording device 102 can include at least two frontal electrodes. The at least two frontal electrodes can be operable to contact a forehead of the patient. In some examples, the one or more electrodes can include a forehead ground electrode. The forehead ground electrode can be operable to reduce electrical noise. The forehead ground electrode can be in contact with a center of the forehead of the subject.

In some examples, the EEG recording device 102 can include one or more occipital electrodes. The one or more occipital electrodes can be operable to record EEG signals arising primarily from the occipital lobe of the subject. For example, the one or more occipital electrodes can be operable to contact the scalp of the subject near the occipital lobe of the subject.

In some examples, the EEG recording device 102 can include one or more reference electrodes. The one or more reference electrodes can provide a comparison point for measuring the electrical potential difference between different electrodes. In some examples, the one or more reference electrodes can include two bilateral reference electrodes on the mastoid bones, which are just behind the ear.

The EEG recording device 102 can be operable to transmit EEG signals, as measured by the one or more electrodes, to the computing device 104. In some examples, the EEG recording device 102 can be operable to measure EEG signals during a plurality of time periods. For example, the EEG recording device 102 can measure a first set of one or more EEG signals at a first time and a second set of EEG signals at a second time. In some examples, the first time, and thereby the first set of one or more EEG signals, can correspond to a time when the subject's eyes are in a closed state. In some examples, the second time, and thereby the second set of EEG signals, can correspond to a time when the subject's eyes are in an open state. In some examples, one or more additional sets of EEG signals can be measured by the EEG recording device 102. The one or more additional sets of EEG signals can correspond to one or more states of the subject (e.g., eyes closed or open) and/or one or more environmental states (e.g., lights on or lights off) and/or different cognitive states (e.g., attentive vs. inattentive, focused vs. daydreaming).

The computing device 104 can be operable to receive the first set of one or more EEG signals and the second set of one or more EEG signals. In some examples, the computing device 104 can further be configured to determine a first power for the first set of one or more EEG signals and a second power for the second set of one or more EEG signals. In some examples, one or more additional sets of EEG signals can be included and a power can be calculated for each additional set of EEG signals.

In some examples, the computing device 104 can be operable to perform a spectral analysis of the first set of one or more EEG signals and the second set of one or more EEG signals focusing on a specific frequency range. For example, the computing device 104 can perform spectral analysis to focus on the alpha band (e.g., 7 Hz to 13 Hz), its sub-bands (e.g., 7-10 Hz and e.g., 10-13 Hz, or other delineations within the overall alpha band) the theta band (e.g., 4 Hz to 7 Hz), the beta band (e.g., 13 Hz to 30 Hz), the gamma band (>30 Hz), and/or other specialized frequency bands or sub-bands.

In some examples, the computing device 104 can be configured to pre-process the EEG signals received from the EEG recording device 102. For example, the computing device 104 can be operable to filter out artifacts or noise from the EEG signals prior to analyzing the EEG signals.

In some examples, the computing device 104 can perform spectral analysis to focus on the frequency range in the alpha band (e.g., 7 Hz to 13 Hz). The computing device 104 can then compute a first alpha power (average of the EEG signals in the alpha frequency range) for the first set of one or more EEG signals. Similarly, the computing device 104 can be operable to perform a spectral analysis of the second set of one or more EEG signals focusing on the frequency range in the alpha band (e.g., 7 Hz to 13 Hz). The computing device 104 can then compute a second alpha power (average of the EEG signals in the alpha frequency range) for the second set of EEG signals.

In some examples, the computing device 104 can further be configured to determine a difference between the first alpha power (i.e., the alpha power associated with the first set of EEG signals) and the second alpha power (i.e., the alpha power associated with the second set of EEG signals). The difference between the first alpha power and the second alpha power can include a raw change or a percentage change. In some examples, such as changing from an eyes-closed to an eyes-open condition, the difference between the first alpha power and the second alpha power can be referred to as alpha reactivity.

In some examples, the computing device 104 can be operable to compare the alpha reactivity for a given subject to a threshold alpha reactivity. In some examples, the threshold alpha reactivity can be based on normalized values relative to demographics or other characteristics. In some examples, the threshold alpha reactivity can include multiple (e.g., one or more) alpha reactivities (e.g., the threshold reactivity can include multiple thresholds that indicate a degree of the subject's attentiveness or inattentiveness). In some examples, the threshold alpha reactivity can be determined based on a number of patients that are clinically diagnosed as attentive or inattentive (e.g., via other testing methods such as a Mini-Mental Status Examination (MMSE)). For example, the alpha reactivities for the patients diagnosed as attentive provide a range of attentive subjects. These alpha reactivities for attentive subjects are higher than alpha reactivities for inattentive subjects. The alpha reactivities for clinically diagnosed attentive subjects can be analyzed to determine an attentive threshold alpha reactivity. Similarly, alpha reactivities for clinically diagnosed inattentive subjects can be analyzed to determine an inattentive threshold alpha reactivity. Further, alpha reactivities for subjects who were clinically diagnosed as attentive before a neurologically impactful event (e.g., administration of anesthesia) and were clinically diagnosed as inattentive after a neurologically impactful event can be analyzed to determine a likelihood to become inattentive based on alpha reactivity.

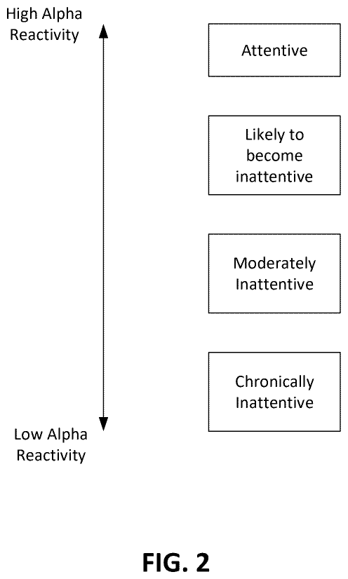

In some examples, the computing device 104 can determine, based on the comparison of the alpha reactivity to the threshold (e.g., a degree of) alpha reactivity, a neurological condition of the subject. For example, the computing device 104 can be operable to determine the attentional control system robustness level of the subject based on the comparison of the alpha reactivity to the threshold (or degree of) alpha reactivity in the population (e.g., threshold alpha reactivities can be based on certain demographics, for example, age). In some examples, when alpha reactivity exceeds (e.g., is higher than) the threshold alpha reactivity, the subject is determined to have a robust attentional control system (e.g., is attentive). In some examples, when the alpha reactivity is lower in degree and/or below the threshold alpha reactivity, the subject is determined to be moderately or chronically inattentive or to have a weakened attentional control system. In some examples, the threshold alpha reactivity can include a scale of thresholds or degrees of alpha reactivities, as illustrated, for example, in FIG. 2.

As illustrated in FIG. 2, the alpha reactivity can be compared to a plurality of alpha reactivity ranges. For example, if the alpha reactivity is below a value of about 2.5 (e.g., eyes-closed alpha power minus eyes-open alpha power), the subject can be diagnosed as chronically inattentive. If the alpha reactivity is between about 2.5 and about 3, the subject can be diagnosed as moderately inattentive. If the alpha reactivity is between about 3 and about 4, the subject can be diagnosed as likely to become inattentive (e.g., the subject is likely to become inattentive due to anesthesia, a surgical procedure, or another neurologically impacting event). If the alpha reactivity is above about 4, the subject can be diagnosed as attentive (e.g., healthy and unlikely to become inattentive due to a neurologically impacting event).

While FIG. 2 illustrates an example of a scale of thresholds or degrees of alpha reactivities, it will be appreciated that other scales can be developed. For example, larger alpha reactivities generally indicate that the subject's attentional control system is functioning properly, whereas lower alpha reactivities generally indicate that the subject's attentional control system is not functioning properly. Alpha reactivities can be different among certain demographics, and the scale of severity of the neurocognitive deficiency can depend on the specific demographic. Therefore, different threshold alpha reactivity scales can be developed based on specific demographics. For example, clinical diagnosis of attentive, inattentive, and newly inattentive subjects can be used to develop the scale for a particular demographic by averaging the alpha reactivity for each group of subjects in the demographic (e.g., attentive, inattentive, and newly inattentive (likely to become inattentive after a neurologic event)). The scale can then be determined based on these average alpha reactivities. Further, additional severity categories can be determined on the scale based on demographic alpha reactivity data. Other approaches can be used to determine threshold alpha reactivities for predicting and/or diagnosing certain neurological disorders (e.g., different tests and clinical assessments to determine subjects with certain neurocognitive disorders or predictors of neurocognitive disorders can be used and compared with those subject's alpha reactivities to determine a scale).

In some examples, the computing device 104 can be further configured to determine alpha reactivities in sub-bands, as variations within certain sub-bands vs. other sub-bands could reflect different functionalities. For example, the computing device 104 can determine an alpha reactivity in a high alpha sub-band (e.g., high-frequency sub-band of 10 Hz to 13 Hz) and a low alpha sub-band (e.g., low-frequency sub-band of 7 Hz to 10 Hz). The alpha reactivity for the sub-bands can be determined in the same manner as the full alpha band reactivity described herein.

In some examples, the computing device 104 can be operable to determine, based on the one or more alpha sub-band reactivities, one or more conditions of the subject. For example, the alpha reactivity in the high alpha sub-band can be indicative of more selective neural systems, such as those involved in anticipating and processing specific sensory input. The alpha reactivity in the low alpha sub-band can be indicative of more diffuse cortical and cortico-thalamic loops regulating global attentional processes, such as alertness.

In some examples, alpha reactivity and/or sub-band alpha reactivities can be used to predict and/or diagnose other neurocognitive conditions. For example, alpha reactivity and/or sub-band alpha reactivities may be effective in predicting and/or diagnosing Alzheimer's Disease (AD); other dementia syndromes; mild cognitive impairment (MCI, a precursor to Alzheimers Disease); Attention Deficit and Hyperactivity Disorder (ADHD); intracranial pathology; depressed cognitive states associated with acute or chronic substance ingestion, pharmaceutical use or intoxication; depressed cognitive states associated with infection or metabolic derangement; Parkinson's Disease (PD); depression; anxiety; and/or other neurological or psychiatric illnesses. These disorders can have alpha reactivity values or sub-band alpha reactivity values associated with them. These values can be determined empirically by looking to a subject population diagnosed and not diagnosed with these disorders and determining a scale similar to the attentiveness scale described herein.

While the system 100 is described as performing neurocognitive weakness analysis focused on the alpha band, it will be appreciated that similar analysis can be conducted in other EEG bands, including theta (4 Hz to 7 Hz), beta (13 Hz to 30 Hz), and gamma (>30 hz). The theta and beta bands can also be used to diagnose and/or predict other cognitive issues, such as MCI, AD, and postoperative cognitive problems. For example, a theta or beta reactivity between the eyes-open and eyes-closed states (e.g., the difference in EEG power) can be indicative of other neurocognitive diseases or disorders, which can be diagnosed using the system 100.

In some examples, the computing device 104 can be operable to guide treatment based on the determined condition of the patient. In some examples, the treatment can include one or more of neurofeedback or biofeedback therapy, family counseling, administration of medications, and/or adjustment to anesthesia-inducing procedures and/or drugs. In addition, the timing of a scheduled surgery can be optimized by considering the likely effects of the surgery (e.g., likelihood that the patient will become newly inattentive or develop another neurocognitive weakness). A physician can be better equipped to make informed treatment plans knowing the risks associated with a surgical procedure for a specific subject and the need for interventions either before or after the surgery.

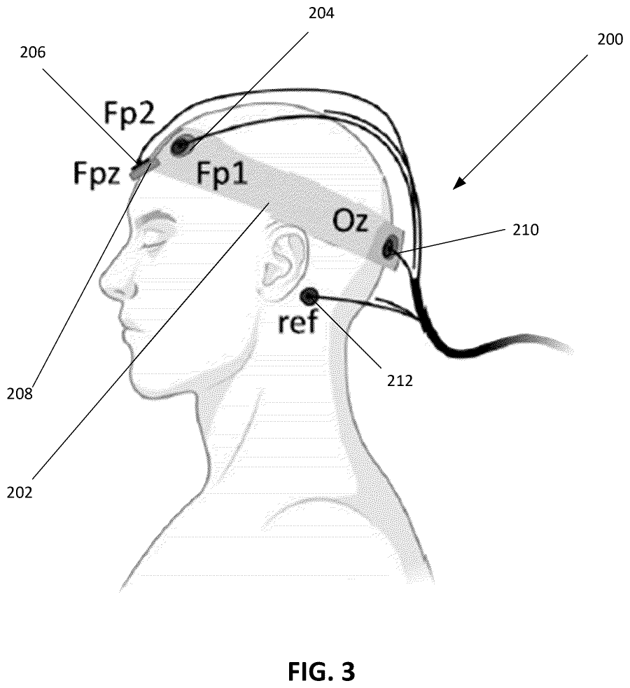

FIG. 3 illustrates an EEG headband 200, which is an example of an EEG recording device 102. The EEG headband 200 can have fewer electrodes than commonly used EEG recording devices and still be effective for the purposes described herein. For example, the EEG headband 200 can include six or even less electrodes. It was found that a minimal number of electrodes (as few as two) can be used in an EEG recording device 102 (e.g., EEG headband 200) and still provide sufficient readings for an alpha reactivity determination.

The EEG headband 200 can include a support structure 202. The support structure 202 can be operable to secure around a head of a subject. In some examples, the support structure 202 can include a soft material for contacting the subject. For example, the support structure 202 can include cotton, cloth, or other cushioning materials. The support structure 202 can be comfortable when worn by the patient.

The EEG headband 200 can further include one or more electrodes for measuring one or more EEG signals of the subject. The one or more electrodes can be coupled to the support structure 202 such that when the support structure is secured to the subject's head, the one or more electrodes are operable to measure one or more EEG signals of the subject.

The EEG headband 200 can further include an amplifier (not shown). The amplifier can be configured to capture, amplify, and convert the electrical signals measured by the one or more electrodes into a useable format for analysis.

In some examples, the EEG headband 200 can include at least two frontal electrodes (e.g., first frontal electrode 204 and second frontal electrode 206). While a first frontal electrode 204 and a second frontal electrode 206 are illustrated in FIG. 3, it will be appreciated that more than two frontal electrodes can be included. The first frontal electrode 204 and the second frontal electrode 206 can be operable to measure one or more EEG signals arising from the frontal lobe of the subject. In other examples, the EEG headband 200 can include a single frontal electrode (e.g., either first frontal electrode 204 or second frontal electrode 206).

In some examples, the one or more electrodes of the EEG headband 200 can include at least one occipital electrode 210. The at least one occipital electrode 210 can be operable to measure EEG signals arising from the occipital lobe of the subject. In some examples, the at least one occipital electrode 210 can include two or more occipital electrodes.

The EEG headband 200 can further include at least one ground electrode 208. In some examples, the at least one ground electrode 208 can be a different frontal electrode, as illustrated, for example, in FIG. 3. In some examples, the at least one ground electrode 208 can be configured to be located in another location of the subject. The at least one ground electrode 208 can be configured to serve as a reference point for the amplifier. The at least one ground electrode 208 can be configured to cancel out noise and ensure accurate measurement of EEG signals. For example, the at least one ground electrode 208 can ground the EEG headband to prevent power line noise from interfering. The at least one ground electrode 208 can be located on the forehead of the subject when the EEG headband 200 is secured on the subject.

The EEG headband 200 can further include one or more reference electrodes, such as on the mastoid. In some examples, the one or more reference electrodes 212 can include bilateral mastoid reference electrodes (e.g., a mastoid reference electrode on each mastoid). In some examples, the one or more reference electrodes 212 can be configured to provide a baseline measure to compare to the active EEG electrodes (e.g., first frontal electrode 204, second frontal electrode 206, and occipital electrode 210).

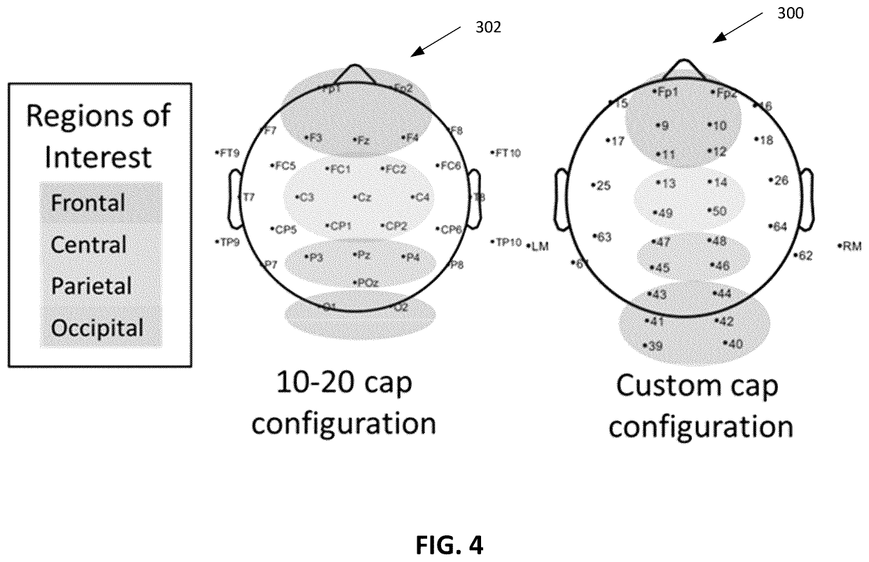

FIG. 4 illustrates a first cap 300 and a second cap 302 that can be used in the system 100 as the EEG recording device 102. The first cap 300 can be operable to record EEG signals of a subject. Each of the first cap 300 and the second cap 302 can have four regions of interest for capturing EEG signals. The regions of interest can include frontal, central, parietal, and occipital areas.

The first cap 300 can include the following electrodes in the regions of interest: frontal region includes electrodes Fp1, Fp2, 9, 10, 11, and 12; central region includes electrodes 13, 14, 49 and 50; parietal region includes electrodes 47, 48, 45, and 46; occipital region includes electrodes 43, 44, 41, 42, 39, and 40.

The second cap 302 can include the following electrodes (from the Standard International 10-20 system) in the regions of interest: frontal region includes electrodes Fp1, Fp2, F3, Fz, and F4; central region includes electrodes FC1, FC2, C3, Cz, C4, CP1, and CP2; parietal region includes electrodes P3, Pz, P4, and POz; occipital region includes electrodes O1 and O2.

Either the first cap 300 or the second cap 302 can be utilized in the system 100 as the EEG recording device 102, as described herein.

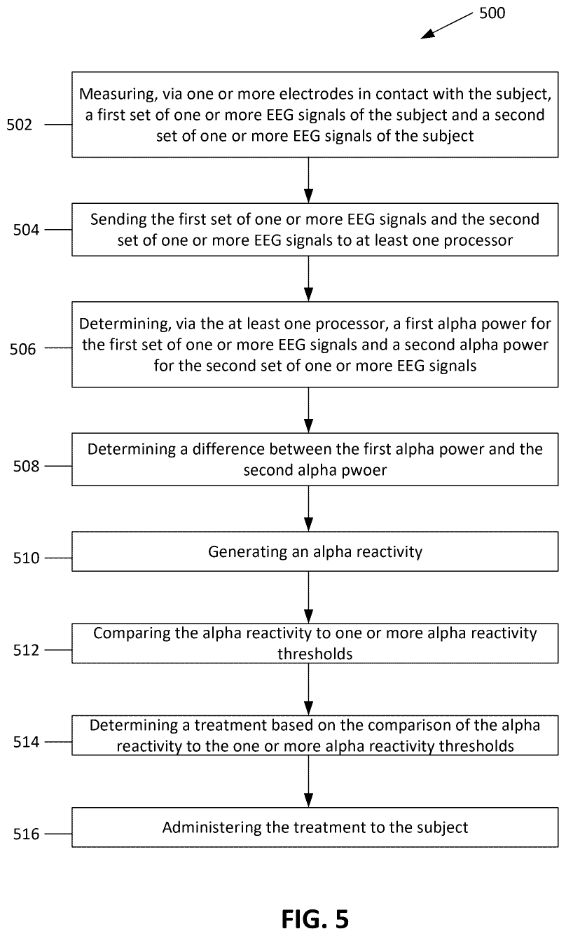

Further provided herein is a method for detecting and/or predicting neurocognitive weakness. FIG. 5 illustrates a flow chart of the method 500. The method 500 can be conducted using the systems described herein.

The method 500 can begin at block 502. At block 502, the method 500 can include measuring, via one or more electrodes in contact with the subject, a first set of one or more EEG signals and a second set of one or more EEG signals. In some examples, the method 500 can first include placing the one or more electrodes on the subject. For example, the EEG headband or EEG caps described herein can first be placed (e.g., secured) on the subject. In some examples, the subject can be instructed to close their eyes before the first set of one or more EEG signals are measured. In some examples, once the subject's eyes are closed, a time marker can be placed in the EEG signal data to mark the beginning of the eyes-closed EEG signal recording. In some examples, the first set of EEG signals can be recorded for about 1 minute to about 2 minutes, about 2 minutes to about 3 minutes, about 3 minutes to about 4 minutes, about 4 minutes to about 5 minutes, or more.

In some examples, once the first set of EEG signals have been recorded (e.g., the time period for recording the first set of EEG signals ends), the subject can be instructed to open their eyes. Once the patient is instructed to open their eyes, a time marker can be placed in the EEG recording data to indicate that the second set of one or more EEG signals is now being recorded (e.g., the second set is recorded with the subject's eyes open). In some examples, the second set of EEG signals can be recorded for about 1 minute to about 2 minutes, about 2 minutes to about 3 minutes, about 3 minutes to about 4 minutes, about 4 minutes to about 5 minutes, or more.

At block 504, the method 500 can include sending the first set of one or more EEG signals and the second set of one or more EEG signals to at least one processor (e.g., the computing device described herein). In some examples, the first set of one or more EEG signals and the second set of one or more EEG signals can be sent to the at least one processor as a single file with the time markers indicating when the first set starts and stops and when the second set starts and stops.

At block 506, the method can include determining, via the at least one processor, a first alpha power for the first set of one or more EEG signals and a second alpha power for the second set of one or more EEG signals. In some examples, determining the first alpha power and the second alpha power can include performing spectral analysis to isolate the EEG signals in a specific frequency. In the case of calculating alpha powers, the EEG signals in the alpha frequency (e.g., 7 Hz to 13 Hz) can be isolated. Once the alpha frequency EEG signals are isolated, an alpha power across the whole head (e.g., the power for all detected EEG signals by the electrodes averaged together) or across certain selected electrodes can be calculated. In this manner, a first alpha power and a second alpha power can be determined.

While the method 500 specifically references alpha as the frequency band of interest, it will be appreciated that the method 500 can similarly isolate other EEG frequency bands. For example, the method 500 can isolate the beta band (13 Hz to 30 Hz) and/or the theta band (4 Hz to 7 Hz).

While the method 500 references the alpha-band in its entirety (i.e., 7 Hz to 13 Hz), it will be appreciated that alpha powers within alpha sub-bands can also be determined. For example, the method 500 can include separating the alpha-band into one or more sub-bands. For example, the alpha-band can be separated into a low sub-band (7 Hz to 10 Hz) and a high sub-band (10 Hz to 13 Hz). The first alpha power and the second alpha power can be determined for each of the low sub-band and the high sub-band.

At block 508, the method 500 can include determining a difference between the first alpha power and the second alpha power. The difference can be determined by subtracting the first alpha power from the second alpha power. In some examples, when the first alpha power is calculated from the first set of EEG signals in the eyes-closed state and the second alpha power is calculated from the second set of EEG signals in the eyes-open state, the difference is the difference in alpha power between the eyes-closed and eyes-open states of the subject.

At block 510, the method 500 can include generating calculating (e.g., generating) an alpha reactivity measure. The alpha reactivity is the difference between the first alpha power in the eyes-closed state and the second alpha power in the eyes-open state, as determined in block 508. Alpha reactivity is a measure of the subject's alpha power during the eyes-closed versus eyes-open states. Alpha reactivity can provide a measure for indicating whether a subject has a neurocognitive weakness or is likely to develop a neurocognitive weakness (e.g., predict future neurocognitive weakness). In some examples, neurocognitive weakness can be defined as whether a patient is attentive, inattentive, or likely to become inattentive. A subject is considered to have a neurocognitive weakness clinically if the subject would be classified as inattentive.

At block 512, the method 500 can include comparing the alpha reactivity to one or more alpha reactivity thresholds or degrees. In some examples, the one or more alpha reactivity thresholds can include a single alpha reactivity threshold. In some examples, the single alpha reactivity threshold is a threshold where if the subject's alpha reactivity is above the threshold, the subject is attentive and unlikely to become inattentive after a neurologically impacting event (e.g., anesthesia administration). If the subject is below the threshold, the subject is either already inattentive or likely to become inattentive after a neurologically impacting event (e.g., anesthesia administration). In some examples, the single threshold is an alpha reactivity of about 4. In some examples, the one or more alpha reactivity thresholds can include a scale of alpha reactivities. For example, if the alpha reactivity is below a value of about 2.5 (e.g., eyes-closed alpha power minus eyes-open alpha power), the subject can be diagnosed as chronically inattentive. If the alpha reactivity is between about 2.5 and about 3, the subject can be diagnosed as moderately inattentive. If the alpha reactivity is between about 3 and about 4, the subject can be diagnosed as likely to become inattentive (e.g., the subject is likely to become inattentive due to anesthesia, a surgical procedure, or another neurologically impacting event). If the alpha reactivity is above about 4, the subject can be diagnosed as attentive (e.g., healthy and unlikely to become inattentive due to a neurologically impacting event). In addition, using different clinical reference cognitive measures, the alpha reactivity measure can be used to predict the likelihood of having neurocognitive weakness or to develop cognitive symptoms after a neurologically impactful event, such as surgery and anesthesia.

Similar to the full alpha band reactivity, the method 500 can include comparing the alpha sub-band reactivities to alpha sub-band reactivity thresholds to determine various conditions of the patient. In some examples, the comparison of the alpha sub-band reactivities to the alpha sub-band reactivity thresholds can provide an indication of one or more conditions of the patient. For example, the one or more conditions can include inattentiveness, delirium, and other neurological conditions. The alpha sub-band reactivities can be calculated in the same manner as the full band alpha reactivity described herein.

At block 514, the method 500 can include determining a treatment based on the comparison of the alpha reactivity to the one or more alpha reactivity thresholds. In some examples, the treatment can include one or more of neurofeedback or biofeedback therapy, family counseling, administration of medications, neuro-modulatory interventions, and/or adjustment of anesthesia-inducing procedures and/or drugs. In some examples, the treatment can consider canceling or modifying a surgery (e.g., choice of anesthesia) if the subject's alpha reactivity indicates that the patient is likely to become inattentive (e.g., have attentional weakness or other cognitive symptoms) after the surgery.

At block 516, the method 500 can include administering the treatment to the patient.

While the method 500 is described in terms of the alpha frequency band, it will be appreciated that other EEG signal frequency bands can be used in the method 500. For example, the method 500 can be performed in substantially the same way in the beta frequency band (13 Hz to 30 Hz), the theta frequency band (4 Hz to 7 Hz), and/or any sub-band in the alpha, beta, or theta frequencies ranges.

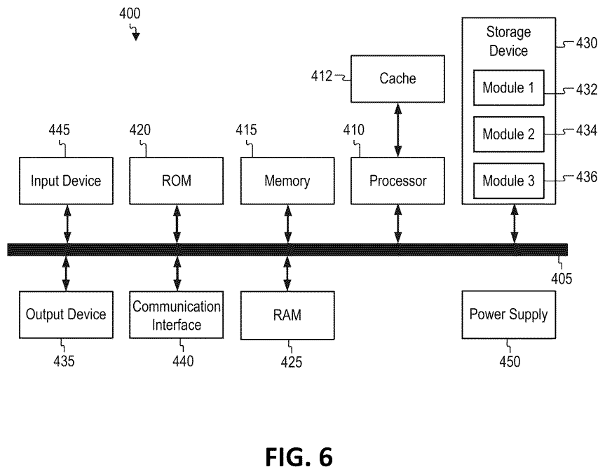

FIG. 6 shows an example of computing system 400, which can be for example any computing device making up various parts of the system (e.g., computing device 104), or any component thereof in which the components of the system are in communication with each other using connection 405. Connection 405 can be a physical connection via a bus, or a direct connection into processor 410, such as in a chipset architecture. Connection 405 can also be a virtual connection, networked connection, edge network connection, or logical connection.

In some embodiments, computing system 400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 400 includes at least one processing unit (CPU or processor) 410 and connection 405 that couples various system components including system memory 415, such as read-only memory (ROM) 420 and random-access memory (RAM) 425 to processor 410. Computing system 400 can include a cache of high-speed memory 412 connected directly with, in close proximity to, or integrated as part of processor 410.

Processor 410 can include any general purpose processor and a hardware service or software service, such as services 432, 432, and 436 stored in storage device 430, configured to control processor 410 as well as a special-purpose processor where software instructions are incorporated into the actual processor design. Processor 410 may essentially be a completely self-contained computing system, containing multiple cores or processors, a bus, memory controller, cache, etc. A multi-core processor may be symmetric or asymmetric.

To enable user interaction, computing system 400 includes an input device 425, which can represent any number of input mechanisms, such as a microphone for speech, a touch-sensitive screen for gesture or graphical input, keyboard, mouse, motion input, speech, etc. Computing system 400 can also include output device 435, which can be one or more of a number of output mechanisms known to those of skill in the art. In some instances, multimodal systems can enable a user to provide multiple types of input/output to communicate with computing system 400. Computing system 400 can include communications interface 420, which can generally govern and manage the user input and system output. There is no restriction on operating on any particular hardware arrangement, and therefore the basic features here may easily be substituted for improved hardware or firmware arrangements as they are developed.

Storage device 430 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 430 can include software services, servers, services, etc., that when the code that defines such software is executed by the processor 410, it causes the system to perform a function. In some embodiments, a hardware service that performs a particular function can include the software component stored in a computer-readable medium in connection with the necessary hardware components, such as processor 410, connection 405, output device 435, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

### EXAMPLES

### Example 1

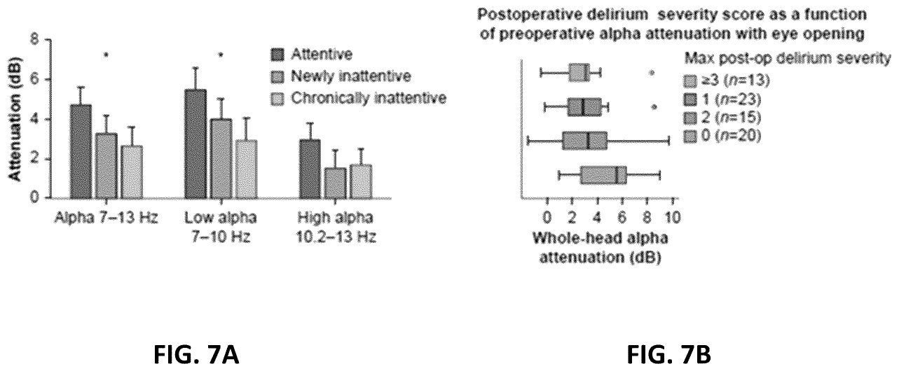

Data from 71 older adults showed a statistically significant association between EEG alpha reactivity (Eyes Closed versus Eyes Open) measured preoperatively and both attentional impairment severity (FIG. 7A) and delirium severity (FIG. 7B) measured postoperatively, after controlling for age, baseline cognition, and multiple comparisons. Further, by separately analyzing the various sub features of delirium, it was found that the delirium severity effect was mainly driven by the sub feature of inattention. These associations also largely held in the frontal region considered alone in multivariable models adjusting for age and baseline cognition score: OR: 0.77, [95% CI; 0.62, 0.96}, p=0.020. Finally, a differential effect between the lower (7-10 Hz) and higher (10-13 Hz) alpha sub-bands suggested the possibility of using EEG reactivity in different alpha sub-bands to dissect more specific neurocognitive processes and impairments.

During the preoperative session, patients undergo point-of-care EEG recording at 1000 Hz sampling rate per channel, with a 0.25-100 Hz preamplifier bandwidth, using a custom active-electrode system (FIG. 3) that includes a custom headband with three recoding sites (two frontal [Fp1 and Fp2] (e.g., first frontal electrode 204 and second frontal electrode 206) along with one occipital site [Oz] (e.g., occipital electrode 210)), one or more reference electrodes 212 (e.g., first bilateral mastoid reference electrode and second bilateral mastoid reference electrode (not shown), and a forehead ground electrode 208 just below FpZ as well as an amplifier in an electrically and acoustically quiet room. Subjects are guided to find a comfortable sitting position, and to refrain from talking or moving during recording (as such movements produce artifacts in the EEG). The start of the eyes-closed segment and each subsequent segment is marked. EEG data is recorded continuously. After three minutes, subjects are instructed to open their eyes. The eyes closed and eyes open segments can be repeated a prescribed number of times to capture an average reactivity and to ensure redundancy in case of excessive noise in one of the eyes-closed/eyes-open pairs.

### Example 2

Up to half of older adults experience perioperative neurocognitive disorders, such as postoperative delirium (POD), which is associated with increased risk for mortality, cognitive decline, and dementia. Even without full-blown delirium, patients with features of delirium, such as inattention, experience impaired recovery and increased mortality risk. Of the cardinal delirium features, inattention is most associated with persistent neurocognitive deficits.

Further, inattention has been associated with poor medication compliance, social isolation, and financial strain, issues that are particularly challenging when superimposed on postoperative recovery. Given the aging surgical population, finding preoperative neural biomarkers that can identify those at highest risk for postoperative inattention would be highly impactful.

Beyond perioperative screening potential, inattention-related neural biomarkers could also inform understanding of neurocognitive mechanisms underlying both POD and one of its cardinal features, inattention. As EEG has been used to investigate vulnerability to other neurocognitive disorders, including POD, and as EEG has sensitivity for attention-related cognitive functions, EEG measures can be evaluated that link brain function and inattention, a core delirium component.

Intraoperative, and to a lesser extent preoperative, EEG has been studied as a POD risk predictor, with variable success. In this example, one core feature of POD, inattention, is focused on. Awake EEG changes under variations in natural brain-state conditions can reveal neurocognitive vulnerability before surgery and anesthesia. Cognitive neuroscience has made extensive links between fundamental attention-related cognitive functions and EEG variations observed in response to changing sensory and cognitive states.

For example, externally directed attention and global arousal inversely correlate with EEG alpha (7-13 Hz) power. In addition, increased EEG alpha power is associated with daydreaming, which is common in awake, task-free, eyes-closed states. In contrast, when the eyes are open, visual stimuli (or the possibility of such) activate thalamic nuclei and cortical structures to induce widespread cortico-cortical and cortico-thalamic interactions as the brain processes incoming visual information. These interactions that occur with opening of the eyes desynchronize EEG alpha oscillations, reducing EEG alpha power. This alpha-power decrease in the eyes-open vs eyes-closed states (i.e. alpha attenuation with eyes opening) remains present in healthy older adults but has been reported to be diminished among those with chronic neurocognitive disorders.

Further, although decreased power in the alpha frequency band (7-13 Hz) likely facilitates neuronal transmission of incoming sensory information, cognitive neuroscientists sometimes subdivide the alpha band because lower and higher alpha sub-bands have been associated with somewhat different attention-related processes. Lower-frequency (e.g. 7-10 Hz) alpha rhythms tend to reflect the more diffuse cortical and cortico-thalamic loops regulating global attentional processes, such as alertness. Higher-frequency (e.g. 10-13 Hz) alpha rhythms have been associated with more selective neural systems, including those involved in anticipating and processing specific sensory input. Thus, inattention pathophysiology and pre-existing neurocognitive vulnerability can be better understood by studying attention-related EEG measures within these alpha sub-bands.

Preoperative cognitive deficits, which are associated with increased postoperative neurocognitive risk, are often unrecognized because clinical costs and time constraints limit in-depth neurocognitive testing. A quick preoperative EEG with a simple ‘eyes-closed eyes-open’ task can reveal a risk for disordered arousal or attentional control systems, allowing clinicians to be alerted to the need for further testing, prophylactic measures, or both. This, in turn, can provide an opportunity to reduce the risk for postoperative inattention, and possibly delirium, pre-emptively. This natural, simple brain-state condition manipulation can also yield insights into the neural mechanisms underlying neurocognitive vulnerability and resilience. Accordingly, data from an older-patient surgery cohort was analyzed to determine whether reduced preoperative alpha attenuation with eyes opening is associated with postoperative inattention (primary outcome), POD measures (secondary outcome), or both.

### Participants

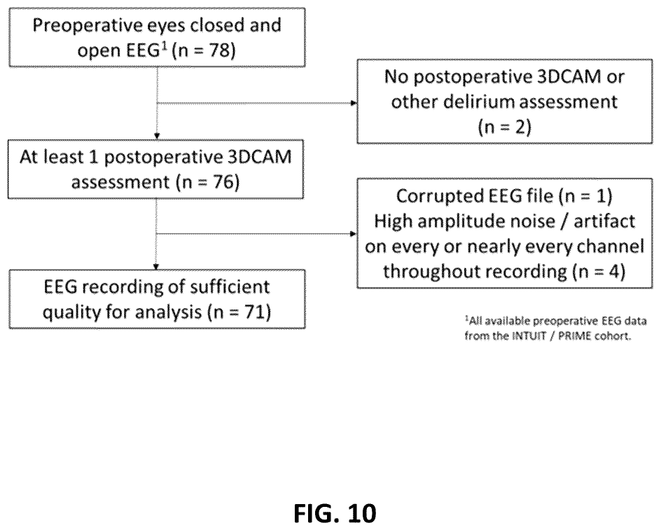

The subjects in this analysis underwent preoperative EEG recordings (n=78). Only a subset of subjects underwent whole-head preoperative EEG. Exclusion criteria included age<60 years, anticipated surgery duration<2 hours, incarceration, inadequate English fluency, and anticoagulant use that would preclude lumbar punctures. There were no cognitive exclusion criteria. Of the 78 subjects who underwent preoperative EEG recordings, two subjects were excluded due to lack of postoperative delirium assessments. Five subjects were excluded due to electromechanical noise or file corruption that precluded analysis. This left 71 subjects for analysis. A clinical STROBE Diagram is shown in FIG. 10.

### Inattention and Delirium Severity Screening

Participants underwent a 3-min diagnostic interview for Confusion Assessment Method-defined delirium (3D-CAM) before surgery (baseline), and after surgery each morning and late afternoon until discharge. Using 3D-CAM feature definitions, ‘attentive’ subjects answered all attention items (#4-7, 16, 17) correctly on both preoperative and postoperative 3D-CAM assessments; ‘inattentive’ subjects had at least one incorrect attention item on at least one postoperative 3D-CAM assessment. Delirium severity was the maximum postoperative score on a 20-point 3D-CAM scale, with higher scores indicating greater delirium severity. Categorical delirium was defined as any positive POD assessment, including nursing assessments.

### Inattention Chronicity

Inattentive subjects were subdivided based on a measure termed ‘inattention chronicity’. ‘Chronically inattentive’ subjects missed at least one attention item both preoperatively and postoperatively. ‘Newly inattentive’ subjects had all attention items correct preoperatively and at least one incorrect attention item on at least one postoperative 3DCAM assessment. Given sample size constraints, patients were not subdivided with improving vs worsening inattention.

### Electroencephalography Recording

Awake, 32-channel, whole-head EEG recording was performed before surgery for 3 min with eyes closed and 3 min with eyes open. An experienced technician monitored EEG recordings, including to ensure patients did not fall asleep. To remove noise and artifacts, acquired EEG data were pre-processed in MATLAB using an EEGLAB toolbox, custom scripts, and contiguous epochs of non-overlapping 3-s windows on each electrode, across all artifact-free data within the recorded 3-min windows. EEG data were re-referenced during pre-processing to the algebraic average of the two mastoid electrodes. The EEG recording devices (e.g., first cap 300 and second cap 302) of FIG. 4 were used in this study.

Once the EEG cap preparation was complete, each subject was asked if they were ready to have their brain data recorded. Each was briefly instructed to try to find a comfortable sitting or reclining position, to try to keep their facial muscles loose and relaxed, and to prepare to record three minutes of eyes-closed then eyes-open data during which they should try to rest comfortably, and refrain from talking or moving. The technician would first let the patient know when to start holding still, and they would then insert a marker into the data at the start of the eyes-open or eyes-shut segment, and finally let the subject know when data recording was finished once three minutes had passed. An experienced EEG technician monitored the recording to help maintain the data quality and to ensure that the patient did not fall asleep. For the first 10 subjects, a tethered, custom EEG cap 3 and recording system (first cap 300 illustrated in FIG. 4) was used. For subsequent subjects, a wireless recording system with a standard international 10-20 EEG cap configuration (second cap 302 illustrated in FIG. 4) was used, which was more convenient and less unwieldy than the tethered system in the hospital setting. FIG. 4 shows the electrode configurations of the caps (first cap 300 and second cap 302). EEG signals were recorded at a 1000 Hz sample rate per channel, with a 0.016-250 Hz passband, online Cz-electrode referencing, and electrode impedances<20 kΩ. One subject inadvertently had EEG signals recorded at a 500 Hz sample rate, which was upsampled to 1000 Hz using linear interpolation in MATLAB prior to analysis. EEG recordings were performed on the day of surgery in the preoperative holding area prior to the administration of sedation (eg, midazolam, fentanyl, etc.). The baseline EEG was not acquired at the same time as the baseline 3D-CAM, which was performed in a separate session. The timing of the EEG depended on the timing of surgery, but typically, given surgical scheduling, occurred in the morning or early afternoon.

### Electroencephalography Alpha Attenuation and Sub-Band Analysis

The alpha power band was defined as 7-13 Hz, given the age of the study population. Non-overlapping ‘lower’ and ‘higher’ alpha sub-bands were defined as 7-10 Hz and 10.2-13 Hz, respectively. To calculate attenuation magnitude with eyes opening in each subject, the eyes-open alpha-band (or alpha sub-band) power was subtracted from the eyes-closed alpha band (or alpha sub-band) power. Raw change was used rather than percentage change because eyes-closed alpha power did not differ significantly between attentive and inattentive subjects.

### Topographic Analysis

Whole-head topographic plots were generated using EEGLAB and custom scripts. To create composite topographic plots, maps were averaged across subjects at each spatial location along the x- and y-dimensions to yield an average map for a given group of subjects, condition, and power band (full alpha band, lower or higher alpha sub-bands). Whole-head analyses were applied to avoid bias for a specific spatial region of interest (ROI), given the frontal electrodes' potential clinical utility vs the parietal-occipital electrodes' potential visual cortical-processing specificity. In follow-up sensitivity analyses, activity in frontal, central, parietal, and occipital ROIs were separately analyzed.

### Statistical Analysis

A statistician, who neither collected nor processed the EEG data, performed analyses using SAS version 9.4, (SAS Institute, Cary, NC, USA). A statistical analysis plan for the primary outcome (inattention) and secondary outcome (delirium severity) was determined before the statistician received EEG data. Data availability determined sample size, as there were no prior studies of preoperative eyes-opening alpha-attenuation to inform prospective power calculations. Baseline characteristics were compared between attentive and inattentive subjects using t-tests or Wilcoxon rank-sum tests for numeric variables and c₂ tests for categorical variables. For univariate associations of whole-head alpha attenuation with inattention, inattention chronicity, delirium severity, and delirium incidence, group-wise t-tests, analyses of variances (ANOVA), or Spearman correlations were used, as appropriate. Delirium severity and inattention chronicity were treated as ordinal variables, with inattention chronicity ranked as attentive, newly inattentive, and chronically inattentive, as described above. Because EEG alpha power decreases with age and preoperative baseline cognition is linked to POD, multivariable regression was used to control for age and Mini-Mental Status Examination (MMSE) score as possible confounders. Baseline cognition was adjusted for using the MMSE, a widely used, easy-to-replicate measure. Two subjects missing MMSE data were excluded from these multivariable analyses. Logistic regression was used to analyze inattention and delirium incidence and proportional-odds regression for inattention chronicity and delirium severity. Analyses were separately applied within each frequency band/sub-band, within the eyes-closed minus eyes-open difference (attenuation) and within the eyes-closed and eyes-open conditions, and within each ROI in sensitivity analyses. All hypotheses were two-sided with significance of alpha less than or equal to 0.05. Unless otherwise noted, all P-values were corrected for multiple comparisons using the Holm method and assessed for normality using the Shapiro-Wilk test.

### Subject Characteristics

Table 1 describes baseline and surgical characteristics for postoperatively attentive (31/71, 43.7%) vs inattentive subjects (40/71, 56.3%). Among inattentive subjects, 18 (25.4% of total) were chronically inattentive and 22 (31.0% of total) were newly inattentive after surgery.

TABLE 1
Cohort description by postoperative attention status. Numeric
variables were summarized with mean (SD) or median (Q1,
Q3) and for categorical variables count (%) was used.

| | Postoperatively | | Postoperatively | | Uncor- |
|---|---|---|---|---|---|
| | attentive | | inattentive | | rected |
| | (N = 31) | | (N = 40) | | P- [symbol] |
| Age (yr) | 66.3 | (4.3) | 70.4 | (6.5) | 0.003* |
| Sex (male), n (%) | 17 | (54.8) | 18 | (45.0) | 0.411^{†} |
| Race, n (%) | | | | | 0.200^{†} |
| Black or African | 3 | (9.7) | 8 | (20.0) | |
| American | | | | | |
| Caucasian/White | 28 | (90.3) | 30 | (75.0) | |
| Other | 0 | (0.0) | 2 | (5.0) | |
| BMI (kg m⁻²), | 31.9 | (5.0) | 27.5 | (4.8) | 0.003* |
| mean (SD) | | | | | |
| Preoperative albumin | 3.9 | (0.4) | 3.8 | (0.6) | 0.395* |
| (g dl−¹) ^{¶}, | | | | | |
| mean (SD) | | | | | |
| Surgical service, | | | | | 0.848^{†} |
| n (%) | | | | | |
| Thoracic surgery | 5 | (16.1) | 10 | (25.0) | |
| General surgery | 8 | (25.8) | 6 | (15.0) | |
| Gynaecology | 4 | (12.9) | 4 | (10.0) | |
| Orthopaedics | 5 | (16.1) | 8 | (20.0) | |
| Plastic surgery | 1 | (3.2) | 1 | (2.5) | |
| Urology | 8 | (25.8) | 11 | (27.5) | |
| Previous cancer | 12 | (38.7) | 17 | (42.5) | 0.747^{†} |
| treatment, n (%) | | | | | |
| ASA physical status | 3 | (2, 3) | 3 | (3, 3) | 0.024^{†} |
| Surgery duration (min) | 129 | (95, 234) | 144 | (108, 175) | 0.685^{†} |
| Years of education | 16 | (14, 18) | 16 | (13, 16) | 0.057^{†} |
| MMSE^{§} | 29 | (28, 30) | 27 | (25, 29) | 0.003^{†} |

*t-test.
^{†}Wilcoxon rank-sum test.
^{¶} Preoperative albumin missing for three attentive, two newly inattentive, and one chronically inattentive patient.
^{§}MMSE missing for one attentive and one chronically inattentive patient. MMSE, Mini-Mental Status Examination. [symbol]
indicates data missing or illegible when filed

Table 2 shows characteristics by postoperative inattention chronicity. Attention status did not significantly differ by surgical duration (Table 1). Age, BMI, ASA physical status, and MMSE score were significantly associated with attention status (Table 1). To assess if poor nutrition or cancer linked lower BMI and inattention, preoperative albumin levels and cancer history were examined; neither was significantly associated with inattention. In multivariate analyses, age and preoperative MMSE was controlled for. Neither BMI nor ASA physical status correlated with alpha attenuation: Spearman's rho (95% confidence interval [CI]): 0.05 (0.18, 0.28); P=0.67 for BMI; and 0.04 (0.20, 0.27); P=0.76 for ASA physical status; thus, no evidence of potential confounding from these factors was found.

TABLE 2
Baseline Characteristics of Inattention Chronicity Cohorts

| | | | Newly | | Chronically | | |
|---|---|---|---|---|---|---|---|
| | Attentive | | Inattentive | | Inattentive | | p |
| | (N = 31) | | (N = 22) | | (N = 18) | | value |
| Age (Years) | 66.3 | (4.3) | 69.3 | (6.2) | 71.6 | (6.9) | 0.008¹ |
| Sex (Male) | 17 | (54.8%) | 10 | (45.5%) | 8 | (44.4%) | 0.712² |
| Race | | | | | | | 0.304² |
| Black or African American | 3 | (9.7%) | 3 | (13.6%) | 5 | (27.8%) | |
| Caucasian/White | 28 | (90.3%) | 18 | (81.8%) | 12 | (66.7%) | |
| Other | 0 | (0.0%) | 1 | (4.5%) | 1 | (5.6%) | |
| BMI | 31.9 | (5.0) | 27.4 | (4.8) | 27.6 | (4.9) | 0.001¹ |
| Preop Albumin* | 3.9 | (0.4) | 3.8 | (0.6) | 3.7 | (0.7) | 0.604¹ |
| Surgical Service | | | | | | | 0.868² |
| Thoracic | 5 | (16.1%) | 5 | (22.7%) | 5 | (27.8%) | |
| General Surgery | 8 | (25.8%) | 4 | (18.2%) | 2 | (11.1%) | |
| Gynaecology | 4 | (12.9%) | 2 | (9.1%) | 2 | (11.1%) | |
| Orthopaedics | 5 | (16.1%) | 6 | (27.3%) | 2 | (11.1%) | |
| Plastic Surgery | 1 | (3.2%) | 0 | (0.0%) | 1 | (5.6%) | |
| Urology | 8 | (25.8%) | 5 | (22.7%) | 6 | (33.3%) | |
| Previous Cancer Dx | 12 | (38.7%) | 12 | (54.6%) | 5 | (27.8%) | 0.219² |
| ASA PS | 3 | [2, 3] | 3 | [2, 3] | 3 | [3, 3] | 0.047³ |
| Surgery Duration (min) | 129 | [95, 234] | 147 | [104, 268] | 143 | [110, 165] | 0.757³ |
| Years of Education | 16 | [14, 18] | 16 | [15, 17] | 14 | [12, 16] | 0.002³ |
| MMSE** | 29 | [28, 30] | 29 | [26, 29] | 26 | [25, 28] | <0.001³ |

P-value key:
¹= ANOVA,
²= Chi-Square,
³= Kruskal Wallis
*Preoperative albumin missing for 3 attentive, 2 newly inattentive, and 1 chronically inattentive patient
**MMSE missing for 1 attentive and 1 chronically inattentive patient

### Alpha Attenuation in Inattentive Subjects

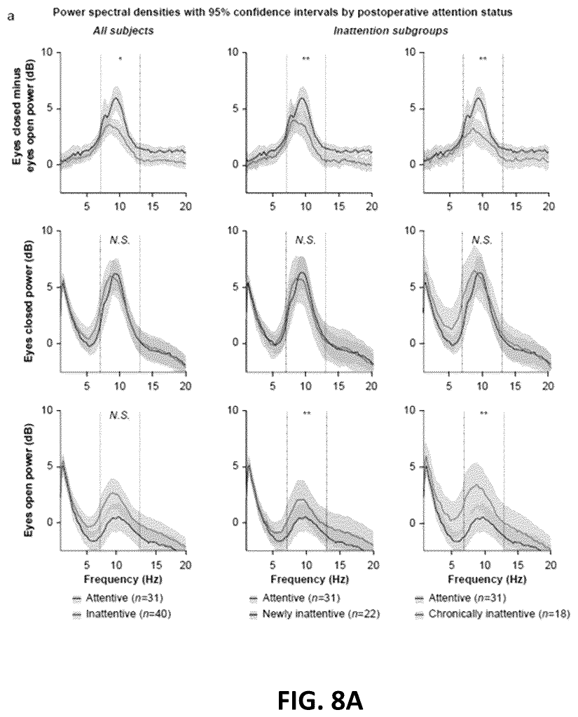

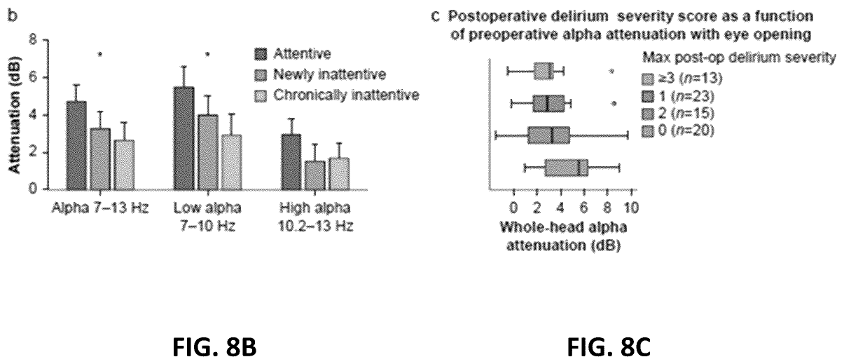

Preoperative whole-head EEG alpha attenuation with eyes opening (eyes-closed minus eyes-open) was significantly greater in subjects that were assessed as being attentive than in those assessed as inattentive, using an age and MMSE-adjusted, multiple comparison-corrected multivariate analyses (FIG. 8A, top left panel, mean with 95% CI shading; Table 3). Alpha power was then analyzed within the individual eyes-closed and eyes-open conditions separately. Under the former, preoperative whole-head alpha power was not associated with postoperative inattention (FIG. 8A, middle left panel, Table 3). Under the eyes-open condition alone, the multivariate relationship between greater alpha power and categorical inattention did not survive multiple-comparison correction (FIG. 8A, bottom left panel, Table 3). The sub-band sensitivity analysis with age- and MMSE-adjusted multivariate models showed less low-band (7-10 Hz) attenuation (eyes-closed minus eyes-open) after correcting for multiple comparisons (Table 3). In the higher (10.2-13 Hz) alpha sub-band, no significant multivariate differences were observed for attenuation, eyes-open, or eyes-closed conditions (Table 3).

TABLE 3
Preoperative alpha attenuation with eyes opening and postoperative attention
status. EEG numeric variables were summarized with mean (SD). Post-hoc
eyes-closed and eyes-open analyses are included for completeness.

| | | | | Multivariate | |
|---|---|---|---|---|---|
| | | | | Age- and MMSE- | |
| | | | | adjusted logistic | |
| | | | Univariate | regression with | |
| Attentive | Inattentive | Difference | t-test | attention status' | |
| (N = 31) | (N = 40) | dB | adjusted | OR | Adjusted |
| dB (SD) | dB (SD) | (95% CI) | P-value² | (95% CI) | P-value^{†} |

Attenuation (eyes closed minus eyes open)

| | | | | | | | | | | |
|---|---|---|---|---|---|---|---|---|---|---|
| Alpha | 4.7 | (2.6) | 3.0 | (2.2) | 1.7 | (0.6, 2.9) | 0.009 | 0.73 | (0.57, 0.94) | 0.038^{‡} |
| (7-13 Hz) | | | | | | | | | | |
| Low alpha | 5.5 | (3.2) | 3.5 | (2.5) | 19 | (0.6, 3.3) | 0.010 | 0.77 | (0.62, 0.95) | 0.038^{‡} |
| (7-10 Hz) | | | | | | | | | | |
| High alpha | 3.0 | (2.3) | 1.6 | (2.1) | 14 | (0.3, 2.4) | 0.010 | 0.77 | (0.59, 1.02) | 0.072 |
| (10.2-13 Hz) | | | | | | | | | | |
| Eyes closed | | | | | | | | | | |
| Alpha | 12.5 | (3.7) | 12.5 | (4.5) | 0.0 | (−2.0, 2.0) | >0.999 | 1.02 | (0.9, 1.16) | >0.999 |
| Low alpha | 10.3 | (4.2) | 10.6 | (4.9) | −0.3 | (−2.5, 1.9) | >0.999 | 1.03 | (0.91, 1.15) | >0.999 |
| High alpha | 7.1 | (3.3) | 7.0 | (4.1) | 0.2 | (−1.6, 2.0) | >0.999 | 1.02 | (0.88, 1.19) | >0.999 |
| Eyes open | | | | | | | | | | |
| Alpha | 7.8 | (3.2) | 9.5 | (4.2) | −1.8 | (−3.6, 0.03) | 0.107 | 1.18 | (1, 1.38) | 0.092 |
| Low alpha | 4.8 | (3.4) | 7.0 | (4.5) | −2.2 | (−4.2, −0.3) | 0.077 | 1.19 | (1.02, 139) | 0.076 |
| High alpha | 4.2 | (3.1) | 5.4 | (3.7) | −1.2 | (−2.8, 0.5) | 0.160 | 1.14 | (0.97, 134) | 0.123 |

*Attention status refers to postoperative ‘attentive’ vs. ‘inattentive’ status.
^{†}Multiple-comparison adjustment performed using the Holm method for three frequency ranges (full alpha, low alpha, and high alpha), only the adjusted p = values are shown.
^{‡}Significant at the P < 0.05 level in multivariate model after multiple-comparison correction.
CI, confidence interval; MMSE, Mini-Mental Status Examination; OR, odds ratio.

Tables 4-6 illustrate the full model results of these analyses.

TABLE 4
All models associated with Alpha-Power Attenuation between the Eyes-Closed and
Eyes-Open States using MMSE as the baseline cognitive-adjustment measure

| | | Inattention | | Inattention | | Delirium | | Delirium | |
|---|---|---|---|---|---|---|---|---|---|
| | | Incidence | | Chronicity | | Incidence | | Severity | |
| EEG | | OR | Uncorr. | OR | Uncorr. | OR | Uncorr. | OR | Uncorr. |
| Metric | Variable | (95% CI) | P-value | (95% CI) | P-value | (95% CI) | P-value | (95% CI) | P-value |
| Whole | Age | 1.08 | 0.1849 | 1.04 | 0.3516 | 0.92 | 0.2748 | 1.05 | 0.2789 |
| Head | | (0.96, 1.2) | | (0.96, 1.14) | | (0.79, 1.07) | | (0.96, 1.14) | |
| (7-13 Hz) | Power | 0.73 | 0.0128 | 0.76 | 0.0096 | 0.98 | 0.9023 | 0.79 | 0.0134 |
| | | (0.57, 0.94) | | (0.62, 0.93) | | (0.72, 1.33) | | (0.65, 0.95) | |
| | MMSE | 0.65 | 0.0115 | 0.63 | 0.0008 | 0.52 | 0.0012 | 0.59 | 0.0001 |
| | | (0.46, 0.91) | | (0.48, 0.83) | | (0.35, 0.77) | | (0.46, 0.76) | |
| Whole | Age | 1.09 | 0.1214 | 1.05 | 0.2423 | 0.92 | 0.2747 | 1.05 | 0.2017 |
| Head | | (0.98, 1.22) | | (0.97, 1.15) | | (0.79, 1.07) | | (0.97, 1.14) | |
| (7-10 Hz) | Power | 0.77 | 0.0133 | 0.77 | 0.0048 | 0.94 | 0.6809 | 0.82 | 0.0141 |
| | | (0.62, 0.95) | | (0.64, 0.92) | | (0.72, 1.24) | | (0.7, 0.96) | |
| | MMSE | 0.65 | 0.0115 | 0.63 | 0.0008 | 0.51 | 0.0012 | 0.6 | 0.0001 |
| | | (0.46, 0.91) | | (0.48, 0.82) | | (0.34, 0.77) | | (0.46, 0.77) | |
| Whole | Age | 1.06 | 0.2797 | 1.04 | 0.3732 | 0.93 | 0.3847 | 1.04 | 0.3455 |
| Head | | (0.95, 1.19) | | (0.95, 1.14) | | (0.79, 1.09) | | (0.96, 1.13) | |
| (10.2-13 | Power | 0.77 | 0.0719 | 0.85 | 0.1667 | 1.14 | 0.4276 | 0.84 | 0.1104 |
| Hz) | | (0.59, 1.02) | | (0.67, 1.07) | | (0.83, 1.57) | | (0.68, 1.04) | |
| | MMSE | 0.66 | 0.0170 | 0.65 | 0.0012 | 0.51 | 0.0013 | 0.61 | 0.0001 |
| | | (0.47, 0.93) | | (0.5, 0.84) | | (0.33, 0.77) | | (0.47, 0.78) | |
| Frontal | Age | 1.08 | 0.1807 | 1.04 | 0.3600 | 0.91 | 0.2559 | 1.04 | 0.3282 |
| ROI | | (0.97, 1.2) | | (0.95, 1.14) | | (0.78, 1.07) | | (0.96, 1.13) | |
| (7-13 Hz) | Power | 0.77 | 0.0195 | 0.79 | 0.0099 | 0.93 | 0.5661 | 0.8 | 0.0073 |
| | | (0.62, 0.96) | | (0.66, 0.94) | | (0.72, 1.2) | | (0.68, 0.94) | |
| | MMSE | 0.64 | 0.0095 | 0.62 | 0.0006 | 0.51 | 0.0011 | 0.58 | 0.0000 |
| | | (0.46, 0.9) | | (0.47, 0.81) | | (0.34, 0.77) | | (0.45, 0.75) | |
| Central | Age | 1.08 | 0.1590 | 1.05 | 0.3124 | 0.91 | 0.2518 | 1.05 | 0.2436 |
| ROI | | (0.97, 1.21) | | (0.96, 1.14) | | (0.78, 1.07) | | (0.97, 1.14) | |
| (7-13 Hz) | Power | 0.75 | 0.0197 | 0.77 | 0.0147 | 0.90 | 0.5295 | 0.82 | 0.0323 |
| | | (0.59, 0.95) | | (0.63, 0.95) | | (0.65, 1.24) | | (0.68, 0.98) | |
| | MMSE | 0.65 | 0.0112 | 0.63 | 0.0008 | 0.51 | 0.0012 | 0.6 | 0.0001 |
| | | (0.47, 0.91) | | (0.48, 0.83) | | (0.34, 0.77) | | (0.47, 0.77) | |
| Parietal | Age | 1.09 | 0.1210 | 1.05 | 0.2475 | 0.92 | 0.2770 | 1.06 | 0.1808 |
| ROI | | (0.98, 1.22) | | (0.97, 1.15) | | (0.79, 1.07) | | (0.97, 1.15) | |
| (7-13 Hz) | Power | 0.78 | 0.0117 | 0.83 | 0.0192 | 0.97 | 0.8191 | 0.86 | 0.0502 |
| | | (0.64, 0.95) | | (0.7, 0.97) | | (0.75, 1.26) | | (0.74, 1) | |
| | MMSE | 0.66 | 0.0151 | 0.64 | 0.0011 | 0.52 | 0.0013 | 0.61 | 0.0001 |
| | | (0.47, 0.92) | | (0.49, 0.84) | | (0.35, 0.77) | | (0.47, 0.78) | |
| Occipital | Age | 1.09 | 0.1403 | 1.05 | 0.2709 | 0.92 | 0.2777 | 1.06 | 0.1848 |
| ROI | | (0.97, 1.21) | | (0.96, 1.14) | | (0.79, 1.07) | | (0.97, 1.15) | |
| (7-13 Hz) | Power | 0.82 | 0.0171 | 0.84 | 0.0131 | 0.99 | 0.9297 | 0.9 | 0.1181 |
| | | (0.7, 0.97) | | (0.73, 0.96) | | (0.79, 1.24) | | (0.79, 1.03) | |
| | MMSE | 0.66 | 0.0201 | 0.65 | 0.0017 | 0.52 | 0.0013 | 0.61 | 0.0001 |
| | | (0.47, 0.94) | | (0.5, 0.85) | | (0.35, 0.77) | 0.0013 | (0.48, 0.79) | |

TABLE 5
All models associated with eyes-open EEG alpha-power considered alone

| | | Inattention | | Inattention | | Delirium | | Delirium | |
|---|---|---|---|---|---|---|---|---|---|
| | | Incidence | | Chronicity | | Incidence | | Severity | |
| EEG | | OR | Uncorr. | OR | Uncorr. | OR | Uncorr. | OR | Uncorr. |
| Metric | Variable | (95% CI) | P-value | (95% CI) | P-value | (95% CI) | P-value | (95% CI) | P-value |
| Whole | Age | 1.09 | 0.1119 | 1.06 | 0.1600 | 0.91 | 0.2456 | 1.07 | 0.1280 |
| Head | | (0.98, 1.21) | | (0.98, 1.16) | | (0.78, 1.07) | | (0.98, 1.16) | |
| (7-13 | Power | 1.18 | 0.0459 | 1.17 | 0.0190 | 0.92 | 0.4303 | 1.12 | 0.0521 |
| Hz) | | (1, 1.38) | | (1.03, 1.33) | | (0.75, 1.13) | | (1, 1.26) | |
| | MMSE | 0.65 | 0.0150 | 0.62 | 0.0007 | 0.5 | 0.0014 | 0.6 | 0.0001 |
| | | (0.46, 0.92) | | (0.48, 0.82) | | (0.33, 0.77) | | (0.47, 0.77) | |
| Whole | Age | 1.09 | 0.1268 | 1.06 | 0.1806 | 0.91 | 0.2568 | 1.06 | 0.1443 |
| Head | | (0.98, 1.21) | | (0.97, 1.16) | | (0.78, 1.07) | | (0.98, 1.15) | |
| (7-10 | Power | 1.19 | 0.0252 | 1.18 | 0.0086 | 0.94 | 0.5300 | 1.12 | 0.0339 |
| Hz) | | (1.02, 1.39) | | (1.04, 1.33) | | (0.78, 1.14) | | (1.01, 1.25) | |
| | MMSE | 0.65 | 0.0151 | 0.62 | 0.0007 | 0.5 | 0.0014 | 0.6 | 0.0001 |
| | | (0.46, 0.92) | | (0.47, 0.82) | | (0.33, 0.77) | | (0.47, 0.77) | |
| Whole | Age | 1.09 | 0.0951 | 1.07 | 0.1359 | 0.91 | 0.2323 | 1.07 | 0.1101 |
| Head | | (0.98, 1.21) | | (0.98, 1.17) | | (0.77, 1.06) | | (0.99, 1.16) | |
| (10.2-13 | Power | 1.14 | 0.1229 | 1.14 | 0.0703 | 0.9 | 0.3397 | 1.11 | 0.1024 |
| Hz) | | (0.97, 1.34) | | (0.99, 1.3) | | (0.72, 1.12) | | (0.98, 1.26) | |
| | MMSE | 0.66 | 0.0153 | 0.63 | 0.0009 | 0.5 | 0.0014 | 0.6 | 0.0001 |
| | | (0.47, 0.92) | | (0.48, 0.83) | | (0.33, 0.77) | | (0.47, 0.77) | |

TABLE 6
All models associated with eyes-closed EEG alpha-power considered alone

| | | Inattention | | Inattention | | Delirium | | Delirium | |
|---|---|---|---|---|---|---|---|---|---|
| | | Incidence | | Chronicity | | Incidence | | Severity | |
| EEG | | OR | Uncorr. | OR | Uncorr. | OR | Uncorr. | OR | Uncorr. |
| Metric | Variable | (95% CI) | P-value | (95% CI) | P-value | (95% CI) | P-value | (95% CI) | P-value |
| Whole | Age | 1.09 | 0.0874 | 1.07 | 0.1316 | 0.91 | 0.2282 | 1.06 | 0.1335 |
| Head | | (0.99, 1.21) | | (0.98, 1.17) | | (0.78, 1.06) | | (0.98, 1.15) | |
| (7-13 | Power | 1.02 | 0.7475 | 1.05 | 0.4378 | 0.92 | 0.3896 | 1.02 | 0.7361 |
| Hz) | | (0.9, 1.16) | | (0.93, 1.17) | | (0.76, 1.12) | | (0.92, 1.13) | |
| | MMSE | 0.67 | 0.0170 | 0.64 | 0.0011 | 0.5 | 0.0013 | 0.61 | 0.0001 |
| | | (0.48, 0.93) | | (0.49, 0.84) | | (0.33, 0.77) | | (0.48, 0.78) | |
| Whole | Age | 1.09 | 0.0918 | 1.07 | 0.1446 | 0.91 | 0.2419 | 1.06 | 0.1375 |
| Head | | (0.99, 1.21) | | (0.98, 1.16) | | (0.78, 1.07) | | (0.98, 1.15) | |
| (7-10 | Power | 1.03 | 0.6605 | 1.04 | 0.4199 | 0.92 | 0.3814 | 1.02 | 0.7046 |
| Hz) | | (0.91, 1.15) | | (0.94, 1.15) | | (0.78, 1.1) | | (0.93, 1.12) | |
| | MMSE | 0.67 | 0.0171 | 0.64 | 0.0011 | 0.5 | 0.0014 | 0.61 | 0.0001 |
| | | (0.48, 0.93) | | (0.49, 0.84) | | (0.33, 0.76) | | (0.48, 0.78) | |
| Whole | Age | 1.1 | 0.0845 | 1.08 | 0.1079 | 0.91 | 0.2450 | 1.07 | 0.1083 |
| Head | | (0.99, 1.22) | | (0.98, 1.18) | | (0.77, 1.07) | | (0.99, 1.16) | |
| (10.2- | Power | 1.02 | 0.7457 | 1.06 | 0.3660 | 0.95 | 0.6415 | 1.04 | 0.4863 |
| 13 Hz) | | (0.88, 1.19) | | (0.93, 1.21) | | (0.77, 1.17) | | (0.92, 1.18) | |
| | MMSE | 0.67 | 0.0167 | 0.64 | 0.0011 | 0.51 | 0.0013 | 0.61 | 0.0001 |
| | | (0.48, 0.93) | | (0.49, 0.84) | | (0.34, 0.77) | | (0.48, 0.78) | |

Sensitivity analysis with a factor-based cognitive adjustment yielded very similar results as illustrated in Table 7.

TABLE 7
EEG Alpha-Power Attenuation between the Eyes-Closed and Eyes-Open States and
inattention juxtaposing the use of MMSE versus the use of a factor-based measure
(continuous cognitive index, CCI) as the baseline cognitive adjustment

| Age- and MMSE-adjusted logistic | | Age- and CCI-adjusted logistic | |
|---|---|---|---|
| regression with attention status¹ | | regression with attention status¹ | |
| OR | Adjusted | OR | Adjusted |
| (95% CI) | p-value² | (95% CI) | p-value² |

Attenuation (Eyes Closed Minus Eyes Open)

| | | | | |
|---|---|---|---|---|
| Alpha (7-13 Hz) | 0.73 (0.57, 0.94) | 0.038* | 0.69 (0.52, 0.93) | 0.034* |
| Low Alpha (7-10 Hz) | 0.77 (0.62, 0.95) | 0.038* | 0.74 (0.58, 0.93) | 0.034* |
| High Alpha (10.2-13 Hz) | 0.77 (0.59, 1.02) | 0.072 | 0.77 (0.55, 1.07) | 0.122 |

¹attention status refers to postoperative “attentive” vs “inattentive” status.
²multiple-comparison adjustment performed using the Holm method for 3 frequency ranges (full alpha, low alpha, and high alpha), only the adjusted p-values are shown

### Inattention Chronicity as a Graduated Function of Alpha Attenuation

To assess inattention chronicity more fully, whole-head power attenuation with eyes opening was plotted (FIG. 8A, top middle and right panels) and quantified for within the full alpha band (7-13 Hz) and within its sub-bands (FIG. 8B). Attenuation in the full alpha-band and in the 7-10 Hz sub-band showed a gradated relationship with inattention chronicity in multivariate proportional-odds regression after correction for age, MMSE score, and multiple comparisons (Table 8). For eyes-open power considered alone, an inverse, gradated relationship with inattention chronicity emerged in the full alpha and 7-10 Hz sub-band (Table 8), although this relationship was weaker than the alpha-attenuation relationship. In the higher (10.2-13 Hz) sub-band, no relationship between attenuation and inattention chronicity (Table 8) survived multivariate analyses.

TABLE 8
Preoperative alpha attenuation with eyes opening and postoperative inattention
chronicity. For numeric variables, we summarize with mean (SD).

| | | | | Multivariate | |
|---|---|---|---|---|---|
| | | | | Age-and MMSE- | |
| | | | | adjusted proportional | |
| | | | | odds regression for | |
| | Newly | Chronically | Univariate | greater inattention | |
| Attentive | inattentive | inattentive | ANOVA | chronicity | |
| (N = 31) | N = 22 | (N = 18) | Adjusted | OR (95% | Adjusted |
| dB (SD) | dB (SD) | dB (SD) | P-value^{†} | CI) | P-value^{†} |

Attenuation (eyes closed minus eyes open)

| | | | | | | | | | | |
|---|---|---|---|---|---|---|---|---|---|---|
| Alpha | 4.7 | (2.6) | 3.2 | (2.2) | 2.6 | (2.2) | 0.024 | 0.76 | (0.62, 0.93) | 0.019^{†} |
| (7-13 Hz) | | | | | | | | | | |
| Low alpha | 5.5 | (3.2) | 4.0 | (2.4) | 2.9 | (2.5) | 0.024 | 0.77 | (0.64, 0.92) | 0.014^{†} |
| (7-10 Hz) | | | | | | | | | | |
| High alpha | 3.0 | (2.3) | 1.5 | (2.3) | 1.7 | (1.8) | 0.037 | 0.85 | (0.67, 1.07) | 0.167 |
| (10.2-13 Hz) | | | | | | | | | | |
| Eyes closed | | | | | | | | | | |
| Alpha | 12.5 | (3.7) | 12.3 | (4.6) | 12.83 | (4.5) | >0.999 | 1.05 | (0.93, 1.17) | >0.999 |
| Low alpha | 10.3 | (4.2) | 10.4 | (4.9) | 10.73 | (5.1) | >0.999 | 1.04 | (0.94, 1.15) | >0.999 |
| High alpha | 7.1 | (3.3) | 6.5 | (4.4) | 7.5 | (3.7) | >0.999 | 1.06 | (0.93, 1.21) | >0.999 |
| Eyes open | | | | | | | | | | |
| Alpha | 7.8 | (3.2) | 9.0 | (4.1) | 10.2 | (4.3) | 0.195 | 1.17 | (1.03, 1.33) | 0.038^{†} |
| Low alpha | 4.8 | (3.4) | 6.4 | (4.4) | 7.8 | (4.7) | 0.138 | 1.18 | (1.04, 1.33) | 0.026^{†} |
| High alpha | 4.2 | (3.1) | 5.0 | (3.7) | 5.8 | (3.9) | 0.288 | 1.14 | (0.99, 1.3) | 0.070 |

*Geater inattention chronicity refers to a longer duration of inattention. Namely, “chronic” inattention status is greater than “newly” inattentive state, which is greater than “attentive” status.
^{†}Multiple-comparison adjustment performed using the Holm method for three frequency ranges. Significance in multivariate model after multiple-comparison correction (for alpha, low alpha, and high alpha) at level of P < 0.05.
CI, confidence interval, MMSE, Mini-Mental Status Examination; OR, odds ratio.

### Delirium Severity and Alpha-Attenuation Magnitude

Whole-head alpha attenuation measured preoperatively was associated with POD severity measured post-operatively (FIG. 8C) in multiple comparison-corrected, multivariate proportional-odds analysis (odds ratio [OR] 0.79, 95% CI: 0.65, 0.95; P=0.04). There was not any significant relationship between preoperative alpha attenuation and the postoperative presence of any delirium feature other than inattention (i.e. altered mental status/fluctuating course, altered level of consciousness, or disorganized thinking) in either univariate analyses or multivariate logistic regression models (Table 9).

TABLE 9
Alpha attenuation in the absence and the presence of non-attention delirium features

| | | | | | Difference | | Multivariate | |
|---|---|---|---|---|---|---|---|---|
| | | | | | (Absent | | Age- and MMSE-adjusted | |
| | Feature | | Feature | | minus | | logistic regression with | |
| | Absent Post-op | | Present Post-op | | present) | Univariate | attention status¹ | |
| | | dB | | dB | dB | T-test | OR | |
| | | mean | | mean | (95% | Adjusted | (95% | Adj. |
| Features | N | (SD) | N | (SD) | CI) | p value¹ | CI) | p-value¹ |

Whole Head Alpha (7-13 Hz) Attenuation (Eyes Closed Minus Eyes Open)

| | | | | | | | | |
|---|---|---|---|---|---|---|---|---|
| Altered | 43 | 3.8 | 28 | 3.6 | 0.2 | >0.999 | 0.98 | >0.999 |
| mental | | (2.5) | | (2.5) | (−1.0, 1.4) | | (0.80, 1.19) | |
| status/ | | | | | | | | |
| fluctuating | | | | | | | | |
| course | | | | | | | | |
| Altered | 56 | 3.8 | 15 | 3.4 | 0.5 | >0.999 | 0.95 | >0.999 |
| level of | | (2.5) | | (2.5) | (−1.0, 1.9) | | (0.74, 1.22) | |
| consciousness | | | | | | | | |
| Disorganized | 69 | 3.8 | 2 | 3.0 | 0.8 | >0.999 | 1.12 | >0.999 |
| Thinking | | (2.5) | | (1.1) | (−2.8, 4.4) | | (0.54, 2.31) | |

¹multiple-comparison adjustment performed using the Holm method across the three non-attention delirium features

Furthermore, after omitting attention items from the delirium severity score, no relationship between alpha attenuation and delirium severity remained (OR 0.96, 95% CI: 0.78, 1.17; P=0.658), further suggesting that the effect on the overall delirium score was driven by the inattention items. Finally, delirium severity was not correlated with whole-head alpha power within the eyes-open or eyes-closed conditions considered alone (Table 10).

TABLE 10
Multivariate models of whole-hear pre-operative alpha attenuation, eyes-open alpha power,
and eyes-closed alpha power as a function of post-operative delirium severity

| | | Multivariate | |
|---|---|---|---|
| | | Age- and MMSE-Adjusted | |
| Univariate | | Proportional Odds Regression | |
| Spearman | | for greater delirium severity | |
| Correlation | Adjusted p- | OR | Adjusted p- |
| (95% CI) | value¹ | (95% CI) | value¹ |

Attenuation (Eyes Closed Minus Eyes Open)

| | | | | | |
|---|---|---|---|---|---|
| Alpha (7-13 Hz) | −0.33 | (−0.52, −0.11) | 0.009 | 0.79 (0.65, 0.95) | 0.040* |
| Low Alpha (7-10 Hz) | −0.34 | (−0.53, −0.12) | 0.009 | 0.82 (0.70, 0.96) | 0.040* |
| High Alpha (10.2-13 Hz) | −0.20 | (−0.41, 0.03) | 0.090 | 0.84 (0.68, 1.04) | 0.110 |

Eyes Closed

| | | | | | |
|---|---|---|---|---|---|
| Alpha | 0.11 | (−0.23, 0.24) | >0.999 | 1.02 (0.92, 1.13) | >0.999 |
| Low Alpha | 0.01 | (−0.23, 0.24) | >0.999 | 1.02 (0.93, 1.12) | >0.999 |
| High Alpha | 0.02 | (−0.21, 0.26) | >0.999 | 1.04 (0.92, 1.18) | >0.999 |

Eyes Open

| | | | | | |
|---|---|---|---|---|---|
| Alpha | 0.22 | (0.01, 0.43) | 0.127 | 1.12 (1.00, 1.26) | 0.104 |
| Low Alpha | 0.24 | (0.01, 0.45) | 0.127 | 1.12 (1.01, 1.25) | 0.102 |
| High Alpha | 0.13 | (−0.10, 0.36) | 0.262 | 1.11 (0.98, 1.26) | 0.104 |

¹multiple-comparison adjustment performed using the Holm method, only the adjusted p-values are shown.
*Indicates significance in multivariate, multiple-comparison-corrected model at level of at p < 0.05

Categorical delirium incidence (11 of 71 subjects) was not significantly associated with any of the EEG measures in this study (Table 11).

TABLE 11
Multivariate models of whole-head pre-operative alpha-power attenuation, eyes-open alpha
power, and eyes-closed alpha power as a function of post-operative delirium (POD) status

| | | | | Multivariate | |
|---|---|---|---|---|---|
| | | | | Age and MMSE Adjusted | |
| Univariate | | | | Logistic Regression delirious | |
| | | | T-test | vs non-delirious | |
| No POD | POD | Difference | Adjusted | OR | Adjusted |
| (N = 60) | (N = 11) | (95% CI) | p-value | (95% CI) | p-value¹ |

Attenuation (Eyes Closed Minus Eyes Open)

| | | | | | | | | | | |
|---|---|---|---|---|---|---|---|---|---|---|
| Alpha | 3.8 | (2.5) | 3.5 | (2.8) | 0.3 | (−1.4, 1.9) | >0.999 | 0.98 | (0.72, 1.33) | >0.999 |
| (7-13 Hz) | | | | | | | | | | |
| Low Alpha | 4.5 | (2.9) | 3.9 | (3.0) | 0.6 | (−1.4, 2.5) | >0.999 | 0.94 | (0.72, 1.24) | >0.999 |
| (7-10 Hz) | | | | | | | | | | |
| High Alpha | 2.1 | (2.2) | 2.5 | (2.5) | −0.3 | (−1.8, 1.2) | >0.999 | 1.14 | (0.83, 1.57) | 0.642 |

(10.2-13 Hz)

Eyes Closed

| | | | | | | | | | | |
|---|---|---|---|---|---|---|---|---|---|---|
| Alpha | 12.6 | (4.2) | 11.9 | (3.7) | 0.7 | (−2.0, 3.4) | >0.999 | 0.92 | (0.76, 1.12) | >0.999 |
| Low Alpha | 10.6 | (4.8) | 9.7 | (3.5) | 0.9 | (−2.2, 3.9) | >0.999 | 0.92 | (0.78, 1.1) | >0.999 |
| High Alpha | 7.1 | (3.7) | 6.9 | (4.5) | 0.2 | (−2.3, 2.7) | >0.999 | 0.95 | (0.77, 1.17) | >0.999 |

Eyes Open

| | | | | | | | | | | |
|---|---|---|---|---|---|---|---|---|---|---|
| Alpha | 8.8 | (4.0) | 8.4 | (2.8) | 0.4 | (−2.1, 2.9) | >0.999 | 0.92 | (0.75, 1.13) | >0.999 |
| Low Alpha | 6.1 | (4.4) | 5.8 | (2.6) | 0.3 | (−2.5, 3.0) | >0.999 | 0.94 | (0.78, 1.14) | >0.999 |
| High Alpha | 4.9 | (3.6) | 4.4 | (3.4) | 0.5 | (−1.8, 2.8) | >0.999 | 0.9 | (0.72, 1.12) | >0.999 |

### Topographic Results: Alpha Attenuation and Inattention

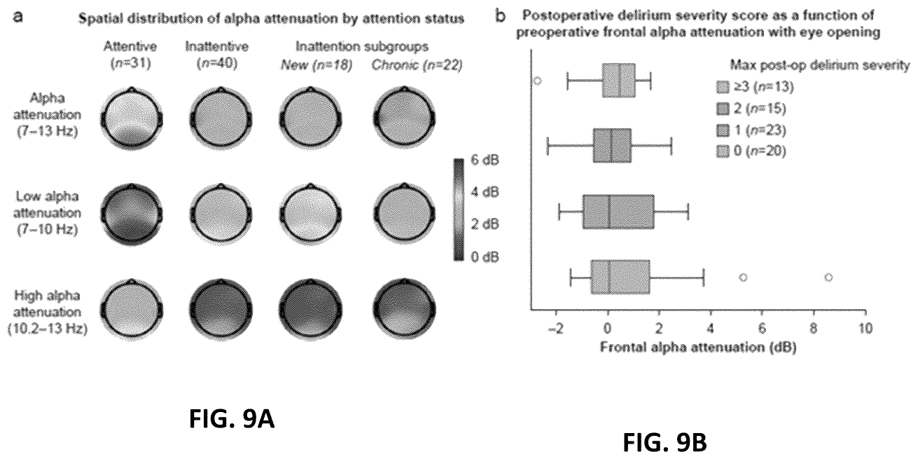

Topographic plots of alpha attenuation with eyes opening (FIG. 9A) for attentive vs inattentive subjects and for the inattention chronicity subgroups showed that alpha power was attenuated across the head, most prominently over the frontal and occipital ROIs. The gradated relationship between alpha attenuation and inattention chronicity in FIG. 8B can be seen in the topographic plots in FIG. 9A. In each ROI, using multivariate logistic regression models, alpha attenuation was significantly associated with attentive vs inattentive status prior to multiple-comparison correction and with inattention chronicity even after multiple-comparison correction (Table 12).

TABLE 12
Relationship between alpha (7-13 Hz) attenuation (eyes closed
minus eyes open) in different topographic regions of interest,
postoperative attention status, and inattention chronicity.

| | Age and MMSE -adjusted logistic | | | Age- and MMSE-adjusted proportional | | |
|---|---|---|---|---|---|---|
| | regression, alpha attenuation, and | | | odds regression for alpha attenuation | | |
| | attentive us inattentive status | | | and greater inattention chronicity | | |
| Region of | OR | Uncorrected | Adjusted* | OR | Uncorrected | Adjusted* |
| interest | (95% CI) | P-value | P-value | (95% CI) | P-value | P-value |
| Frontal | 0.77 | 0.020 | 0.051 | 0.79 | 0.010 | 0.040^{†} |
| | (0.62, 0.96) | | | (0.66, 0.94) | | |
| Central | 0.75 | 0.020 | 0.051 | 0.77 | 0.015 | 0.040^{†} |
| | (0.59, 0.95) | | | (0.63, 0.95) | | |
| Parietal | 0.78 | 0.012 | 0.047^{†} | 0.83 | 0.019 | 0.040^{†} |
| | (0.64, 0.95) | | | (0.7, 0.97) | | |
| Occipital | 0.82 | 0.017 | 0.051 | 0.84 | 0.013 | 0.040^{†} |
| | (0.7, 0.97) | | | (0.73, 0.96) | | |

*Multiple-comparison adjustment for four regions of interest, performed using the Holm method.
^{†}Significance at the P < 0.05 level in multivariate model after multiple-comparison correction.
CI, confidence interval, MMSE, Mini-mental Status Examination; OR, odds ratio.

### Topographic Results: Alpha Attenuation and Delirium Severity

In an age- and MMSE-adjusted proportional-odds analysis within the various ROIs (Table 13), only frontal alpha attenuation remained significantly associated with delirium severity after multiple-comparison adjustment (OR 0.8, 95% CI: 0.68, 0.94; P=0.03; see FIG. 9B for visualization). Alpha attenuation in the occipital ROI was not associated with delirium severity even before multiple-comparison correction (OR 0.90, 95% CI: 0.79, 1.03; P=0.12, uncorrected). Thus, although alpha power with eyes opening was most attenuated occipitally (FIG. 9A), alpha attenuation magnitude in the frontal, but not occipital, region showed more of a potential relationship with delirium severity (Table 13), driven by the strong relationships observed between inattention and alpha attenuation.

TABLE 13
Multivariate models of pre-operative alpha attenuation
by spatial region and post-operative delirium severity

| | | | Multivariate | | |
|---|---|---|---|---|---|
| | | | Age- and MMSE- Adjusted | | |
| | Univariate | | Proportional Odds Regression | | |
| | Spearman | | for greater delirium severity | | |
| | Correlation | Adjusted | OR | | Adjusted |
| | (95% CI) | p-value¹ | (95% CI) | | p-value¹ |
| Frontal | −0.33 (−0.52, −0.10) | 0.019 | 0.8 | (0.68, 0.94) | 0.02* |
| Central | −0.29 (−0.49, −0.06) | 0.023 | 0.82 | (0.68, 0.98) | 0.097 |
| Parietal | −0.31 (−0.51, −0.08) | 0.023 | 0.86 | (0.74, 1) | 0.100 |
| Occipital | −0.30 (−0.50, −0.07) | 0.023 | 0.9 | (0.79, 1.03) | 0.118 |

### Discussion

Preoperative eye-opening EEG alpha attenuation was associated with postoperative inattention, the primary outcome measure, and with delirium severity, the secondary measure. Further, EEG alpha attenuation with eyes opening showed a significant, ordered relationship between attentive, newly inattentive, and chronically inattentive subjects. This suggests a previously unappreciated inattention-susceptibility spectrum. Further, new onset postoperative inattention may occur without known preoperative attention deficits. Thus, preoperative neural measures (i.e. EEG) might reveal vulnerability for attention deficits that can occur after stressor events such as anesthesia and surgery. Even chronically inattentive patients had normal preoperative MMSE scores. The relationship between alpha attenuation and inattention remained after adjusting for preoperative MMSE scores (and in sensitivity analyses with alternative factor analysis-based cognitive adjustment, see Table 7).

Taken together, these results suggest that lower preoperative alpha-attenuation magnitude with eyes opening may offer predictive information about vulnerability for postoperative neurocognitive dysfunction beyond what common clinical cognitive screening tests, such as the MMSE, can show. Moreover, preoperative measures of brain activity, as examined with EEG, can provide neural-level insight into the mechanisms underlying such vulnerability.

Based on prior EEG studies in healthy adults, diminished preoperative alpha attenuation with eyes opening as possibly reflecting impaired alpha suppression, with this impairment being associated with decreased arousal attentiveness, likely leading greater inattention to external stimuli. Furthermore, the eyes-closed/eyes-open paradigm captured visual reactivity with greater sensitivity than the stand-alone eyes-open condition, as the stronger attenuation ORs suggest. The attenuation measure used adjusts for baseline eyes-closed alpha power and thus better anchors the EEG metrics to each patient individually.

This simple eyes-closed/eyes-open paradigm allows for examination of different cognitive functions linked to different sub-bands of the alpha frequency range. Lower-frequency alpha rhythms are thought to reflect more global attentional processes, such as alertness, whereas higher-frequency alpha rhythms have been more associated with selective neural system modulation, such as specific sensory information processing. The 3D-CAM attention questions require general attentiveness and alertness but do not necessarily require more selective attentional processing.

Consistent with this perspective, preoperative alpha attenuation in the lower sub-band was more strongly associated with postoperative inattention measured via 3D-CAM than was attenuation in the higher alpha sub-band.

Further detail of the study of example 2 is provided below.

### EEG Recording

Once the EEG cap preparation was complete, each subject was asked if they were ready to have their brain data recorded. Each was briefly instructed to try to find a comfortable sitting or reclining position, to try to keep their facial muscles loose and relaxed, and to prepare to record three minutes of eyes-closed then eyes-open data during which they should try to rest comfortably, and refrain from talking or moving, as such movements would increase nonneural artifacts in the EEG. The technician would first let the patient know when to start holding still, would then insert a marker into the data at the start of the eyes-open or eyes-shut segment, and finally let the subject know when data recording was finished once three minutes had passed. An experienced EEG technician monitored the recording to help maintain the data quality and to ensure that the patient did not fall asleep. For the first 10 subjects, a tethered, custom EEG cap 3 and recording system (first cap 300 of FIG. 4) was used. For subsequent subjects, a wireless recording system with a standard international 10-20 EEG cap configuration (second cap 302 of FIG. 4) was used. FIG. 4 shows the electrode configurations of the caps. EEG signals were recorded at a 1000 Hz sample rate per channel, with a 0.016-250 Hz passband, online Cz-electrode referencing, and electrode impedances<20 kΩ. One subject inadvertently had EEG signals recorded at a 500 Hz sample rate, which was upsampled to 1000 Hz using linear interpolation in MATLAB (The MathWorks, Inc., Natick, MA, USA) prior to analysis. EEG recordings were performed on the day of surgery in the preoperative holding area prior to the administration of sedation (eg, midazolam, fentanyl, etc.). The baseline EEG was not acquired at the same time as the baseline 3DCAM, which was performed in a separate session. The timing of the EEG depended on the timing of surgery, but typically, given surgical scheduling, occurred in the morning or early afternoon.

### EEG Pre-Processing

The EEG pre-processing was performed on the three-minute segments of eyes-closed and eyes-open EEG starting from time markers placed in the dataset by the recording technician. To remove noise and artefact, the acquired EEG data were pre-processed in MATLAB using the EEGLAB toolbox and custom scripts. EEG recordings were re-referenced to the algebraic average of the two mastoid electrodes. In one subject, one of the two mastoid electrodes was missing, and the mirror image mastoid electrode was interpolated in place of the missing channel. Next, raw 3-minute EEG signals were bandpass-filtered using two hamming-tapered filters (high pass [1 Hz half-amplitude cut-off and 0.08 Hz transition] and low pass [50 Hz half-amplitude cut-off and 1.6 Hz transition]). An expert observer blinded to patient post-surgical outcomes examined the raw EEG data for recording-long artefacts. Channels containing artefacts precluding analysis were interpolated using spherical spline interpolation if adjacent channels had sufficiently clean data. Otherwise, channels with such artefacts were excluded.

Overall, a two-stage method was employed to remove data artefacts. The first phase, based on independent component analysis (ICA), was used to remove artefacts like blinks and eye-movements, given that these artefacts have a stable topography over the course of a recording session. The second phase used an automated-threshold method to reject epochs containing artefacts that could not be cleaned out of the data.

More specifically, prior to the first phase of data cleaning, in preparation for ICA and artefact rejection, each electrode's signal was divided into contiguous epochs of non-overlapping 3-second windows; epochs containing channels with voltages exceeding ±100 μV from the mean were excluded for the purpose of deriving the ICA components. ICA components consisting of muscular, ocular, or cardiac artefacts were identified. Then that ICA decomposition found on the clean subset of the data was applied back to all of the epochs, and the artefactual components were removed. Again, channels in each epoch containing voltages exceeding ±100 μV from the mean were marked. Epochs containing fewer than five marked channels were retained after interpolating those channels just within the epoch; epochs containing five or more marked channels were removed from analysis (typically only a few epochs were rejected per subject). All subjects had usable data channels in all the four regions of interest (ROIs) (i.e., frontal, central, parietal, occipital). Both the eyes-closed and eyes-open preoperative recordings underwent identical pre-processing.

### EEG Spectral Analysis

The power spectrum for each electrode, subject, and condition (eyes open or eyes closed) was computed in MATLAB using EEGLAB and fast Fourier transforms (FFTs). The FFT was performed from 0.25 Hz to 50 Hz with a 5000-sample sliding window, at the 1000-Hz sample rate with 50% overlap (2500 samples), yielding spectral resolution to 0.2 Hz. These per electrode, subject, and condition spectra formed the basis for all subsequent analyses. All available data for a given subject/condition (i.e., full 3 minutes in each condition) were used to generate the single resulting spectrum. For the whole-head analyses, the power-by-frequency spectra for all electrode channels present (up to all 32) were averaged together with equal weight per electrode to generate a mean spectrum across the head. To calculate the average spectrum for a particular spatial ROI for a given subject and condition, the spectra of all electrodes in the ROI were averaged with equal weight per electrode across a given frequency to yield mean power (dB) as a function of frequency (Hz). Percent power change was not used with eye opening/closing because the percent power change must be calculated from the raw power calculations. Further, supplementary analysis was performed with serial t-tests between groups to determine whether there were significant baseline differences in whole-head eyes-closed EEG power among the groups.

### EEG Alpha Power Calculations and Attenuation

The average spectrum was calculated across all electrodes in each ROI (or across all electrodes, up to 32, for the whole-head condition) for a given subject/condition. Then, the logarithmic power spectrum was converted to squared voltage per hertz and integrated using the trapezoidal rule to determine the total power in the alpha band. This total power was then converted back to logarithmic power for ease of comparison. Alpha power, thus, was the total power (in dB) in the 7-13 Hz alpha band. To calculate alpha attenuation, the total eyes-open alpha power was subtracted from the eyes-closed alpha power (in dB). Low sub-band and high sub-band alpha power in each of the two conditions were calculated similarly to the overall (full-band) alpha power. Again, alpha attenuation in the eyes-open vs eyes-closed conditions for the alpha sub-bands was calculated in the same manner as for the full alpha band.

### Topographical Analysis

To allow for proper comparisons among subjects whose EEGs were recorded using different caps, electrode locations were remapped to a new single montage by interpolation. A whole head topographic mesh was generated for each subject using each of the following frequency bands: alpha power (primarily), and low and high alpha (secondarily). At this point, all subjects had maps with the same spatial locations represented, regardless of initial recording cap. Topographic maps power attenuation were evaluated with eye opening (eyes open vs. eyes closed power, multiplied by −1 to yield the magnitude of attenuation).

### Eyes-Closed Whole-Head EEG Alpha Power

In the eyes-closed condition, there were no significant differences between the attentive and inattentive groups in the whole-head EEG alpha power. Thus, there were no baseline eyes-closed whole-head EEG differences among the attention cohorts and that the use of eyes closed minus eyes open alpha power remained an appropriate means of reporting alpha attenuation.

### Power Analysis

The power analysis was based on a simulation of two numerical variables to determine the sample size necessary to detect a Spearman correlation coefficient of +/−0.3 or greater for a hypothetical association between whole head EEG alpha attenuation and delirium severity.

### Univariate Analysis

For univariable associations of whole-head alpha attenuation with inattention, inattention chronicity, delirium severity, and delirium incidence, group-wise t-tests, analyses of variances (ANOVAs), or Spearman correlations, as appropriate were used. An in-depth discussion of normality assessments and the alternative univariate techniques used for non-normal distributions in exploratory, post-hoc analyses outside of the alpha frequency range is further provided herein.

### Delirium Severity Analysis

Univariate analysis with alpha attenuation was conducted via Spearman correlation as delirium severity scores were right skewed. Proportional odds regression was utilized to account for the ordinal nature of the severity score and adjust for the potential confounding effects of age and MMSE. The score test was used to evaluate the proportional odds assumption, and if that test indicated a violation of that assumption, the sparse high severity levels were collapsed until the proportional odds assumption was met. This preserved the ordinal nature and retained as many levels of the severity outcome as possible while ensuring all model assumptions were met. Once scores of four and above were combined, the model passed the score test for proportional odds. Once the model passed the score test for proportional odds, no additional combinations of levels were performed, to ensure that the data continued to drive the analytic approach.

### Normality Assessments

Shapiro-Wilks tests and visual inspection of quantile-quantile plots were used to assess normality. Alpha attenuation (eyes closed minus eyes open) was tested for normality with the Shapiro-Wilks test and passed, and hence parametric comparisons were used. Alpha, its lower and higher sub-bands, and beta (13-30 Hz) all passed the Shapiro-Wilks tests for normality; however, delta (1-4 Hz), theta (4-7 Hz), and gamma (30-50 Hz) did not pass the Shapiro-Wilks tests for normality. Thus, in the non-alpha exploratory post-hoc analyses (described further herein) means, mean differences, t-tests, and ANOVAs were used for beta, just as for alpha, but medians, Hodges-Lehmann (HL) location shifts, Wilcoxon rank sum results, and Kruskal Wallis results were used for the other frequency bands that did not pass Shapiro-Wilks tests for normality.

### Factor-Based Adjustments for Baseline Cognition

As an alternative to MMSE-adjusted baseline cognition, factor-based analysis can be used via the continuous cognitive index (CCI), (i.e., a means of characterizing cognitive function). As a dataset-specific and cognitive-battery-specific index, CCI uses factor analysis with oblique rotation, which is a means of linear data transformation that allows for correlated factors. Previously, factor analysis revealed that five factors accounted for 80% of the variability in full battery of cognitive tests performed at baseline. These factors were 1) structured verbal memory; 2) unstructured verbal memory; 3) visual memory; 4) executive function; and 5) attention/concentration. A 14-item test battery drawn from the larger set of 9 cognitive tests performed for all subjects was used to calculate an average score for each of the five factors. For the “structured verbal memory” factor, 3 items from the Hopkins Verbal Learning test were used. For the “unstructured verbal memory” factor, 4 items from the Randt Short-Story memory test were used. For the “visual memory” factor, 2 items form the Wechsler Memory-Scale-Revised Modified Visual Reproduction Test (a subtest from the larger Wechsler Adult Intelligence Scale-Revised) were used. For the “executive function” factor, a combination of items from Trail Making Tests A and B and the digit symbol test (a subtest from the larger Wechsler Adult Intelligence Scale-Revised) were used. For the “attention/concentration” factor, 2 items from the digit span test (a subtest from the larger Wechsler Adult Intelligence Scale-Revised) were used.

The CCI is the average of the five domain scores calculated via weighted factor analysis on the baseline scores of a large patient cohort. Because factor analysis requires large samples, this study used a combination of all subjects from a larger study (N=196), including subjects who did not undergo preoperative whole head EEG recording and, thus, were not part of this study; and subjects from the another study (N=134) study, which had similar inclusion criteria and a similar preoperative test battery. Thus, factor analysis was performed on a total of 330 subjects which generated scores across the 5 factor domains and provided relative weights for comparison across the cohort of 71 subjects in this study. The CCI scores calculated for individual subjects in this study, thus, are not absolute values or pure averages of the items or factor but rather are normalized relative to the overall cohort of 330 INTUIT and MADCO-PC subjects.

Table 7 directly compares results from the age and MMSE-adjusted multivariable models for the primary outcome (the relationship between whole head alpha attenuation and attentive versus inattentive status), in the left-sided columns, with the age and CCI-adjusted multivariable models for the primary outcome, in the right-sided columns. The results using models with two different baseline cognition adjustment methods are nearly identical.

Table 14 presents the 28 alpha power attenuation multivariable models in Table 4, except using CCI rather than MMSE for baseline cognitive adjustment. The results for alpha attenuation across the whole head and its regions of interest are similar for all four outcomes: inattention incidence, inattention chronicity, delirium incidence, and delirium severity.

TABLE 14
All models associated with EEG Alpha-Power Attenuation between the Eyes-Closed and Eyes-
Open States using Factor-based cognitive (continuous cognitive index, CCI) rather than MMSE

| | | Inattention | | Inattention | | Delirium | | Delirium | |
|---|---|---|---|---|---|---|---|---|---|
| | | Incidence | | Chronicity | | Incidence | | Severity | |
| EEG | | OR | Uncorr. | OR | Uncorr. | OR | Uncorr. | OR | Uncorr. |
| Metric | Variable | (95% CI) | P-value | (95% CI) | P-value | (95% CI) | P-value | (95% CI) | P-value |
| Whole | Age | 1.00 | 0.9840 | 0.99 | 0.8015 | 0.87 | 0.1224 | 1.01 | 0.8160 |
| Head | | (0.88, 1.14) | | (0.89, 1.09) | | (0.73, 1.04) | | (0.93, 1.10) | |
| (7-13 Hz) | Power | 0.69 | 0.0142 | 0.73 | 0.0069 | 1.08 | 0.6466 | 0.81 | 0.0290 |
| | | (0.52, 0.93) | | (0.58, 0.92) | | (0.79, 1.48) | | (0.67, 0.98) | |
| | CCI | 0.04 | 0.0005 | 0.04 | <0.0001 | 0.06 | 0.0005 | 0.16 | 0.0001 |
| | | (0.01, 0.24) | | (0.01, 0.17) | | (0.01, 0.30) | | (0.07, 0.39) | |
| Whole | Age | 1.01 | 0.8263 | 1.00 | 0.9914 | 0.87 | 0.1251 | 1.02 | 0.6966 |
| Head | | (0.89, 1.15) | | (0.90, 1.10) | | (0.73, 1.04) | | (0.93, 1.11) | |
| (7-10 Hz) | Power | 0.74 | 0.0112 | 0.74 | 0.0025 | 1.02 | 0.8893 | 0.83 | 0.0229 |
| | | (0.58, 0.93) | | (0.61, 0.9) | | (0.78, 1.33) | | (0.71, 0.97) | |
| | CCI | 0.04 | 0.0005 | 0.04 | <0.0001 | 0.07 | 0.0006 | 0.16 | 0.0001 |
| | | (0.01, 0.23) | | (0.01, 0.16) | | (0.01, 0.31) | | (0.06, 0.39) | |
| Whole | Age | 0.99 | 0.9253 | 0.99 | 0.8149 | 0.88 | 0.1681 | 1.01 | 0.7733 |
| Head | | (0.87, 1.13) | | (0.89, 1.10) | | (0.73, 1.06) | | (0.93, 1.11) | |
| (10.2-13 | Power | 0.77 | 0.1217 | 0.84 | 0.2013 | 1.28 | 0.2064 | 0.89 | 0.2749 |
| Hz) | | (0.55, 1.07) | | (0.64, 1.10) | | (0.87, 1.89) | | (0.71, 1.10) | |
| | CCI | 0.05 | 0.0006 | 0.06 | <0.0001 | 0.06 | 0.0005 | 0.17 | 0.0001 |
| | | (0.01, 0.28) | | (0.02, 0.20) | | (0.01, 0.28) | | (0.07, 0.41) | |
| Frontal | Age | 1.00 | 0.9881 | 0.98 | 0.7294 | 0.87 | 0.1249 | 1.01 | 0.9087 |
| ROI | | (0.88, 1.14) | | (0.89, 1.09) | | (0.73, 1.04) | | (0.92, 1.1) | |
| (7-13 Hz) | Power | 0.73 | 0.0156 | 0.74 | 0.0032 | 0.99 | 0.9215 | 0.81 | 0.0123 |
| | | (0.57, 0.94) | | (0.61, 0.91) | | (0.75, 1.30) | | (0.69, 0.96) | |
| | CCI | 0.04 | 0.0004 | 0.04 | <0.0001 | 0.07 | 0.0005 | 0.15 | <0.0001 |
| | | (0.01, 0.23) | | (0.01, 0.15) | | (0.01, 0.31) | | (0.06, 0.36) | |
| Central | Age | 1.01 | 0.8581 | 1.00 | 0.9674 | 0.87 | 0.1247 | 1.02 | 0.6867 |
| ROI | | (0.89, 1.15) | | (0.90, 1.1) | | (0.73, 1.04) | | (0.93, 1.11) | |
| (7-13 Hz) | Power | 0.74 | 0.0334 | 0.78 | 0.0226 | 1.03 | 0.8604 | 0.85 | 0.0891 |
| | | (0.56, 0.98) | | (0.62, 0.97) | | (0.75, 1.41) | | (0.71, 1.02) | |
| | CCI | 0.05 | 0.0007 | 0.05 | <0.0001 | 0.06 | 0.0006 | 0.17 | 0.0001 |
| | | (0.01, 0.27) | | (0.01, 0.19) | | (0.01, 0.31) | | (0.07, 0.41) | |
| Parietal | Age | 1.01 | 0.8804 | 1.00 | 0.9835 | 0.87 | 0.1240 | 1.02 | 0.6416 |
| ROI | | (0.89, 1.15) | | (0.90, 1.1) | | (0.73, 1.04) | | (0.94, 1.11) | |
| (7-13 Hz) | Power | 0.75 | 0.0134 | 0.81 | 0.0150 | 1.04 | 0.7689 | 0.87 | 0.0761 |
| | | (0.60, 0.94) | | (0.68, 0.96) | | (0.80, 1.35) | | (0.75, 1.01) | |
| | CCI | 0.04 | 0.0007 | 0.05 | <0.0001 | 0.06 | 0.0006 | 0.17 | 0.0001 |
| | | (0.01, 0.25) | | (0.01, 0.18) | | (0.01, 0.31) | | (0.07, 0.41) | |
| Occipital | Age | 1.01 | 0.8464 | 1.00 | 0.9305 | 0.87 | 0.1217 | 1.02 | 0.6468 |
| ROI | | (0.89, 1.15) | | (0.90, 1.1) | | (0.73, 1.04) | | (0.94, 1.11) | |
| (7-13 Hz) | Power | 0.80 | 0.0208 | 0.82 | 0.0106 | 1.05 | 0.7031 | 0.91 | 0.1732 |
| | | (0.66, 0.97) | | (0.70, 0.95) | | (0.83, 1.32) | | (0.8, 1.04) | |
| | CCI | 0.04 | 0.0006 | 0.05 | <0.0001 | 0.06 | 0.0006 | 0.17 | 0.0001 |
| | | (0.01, 0.26) | | (0.01, 0.18) | | (0.01, 0.30) | | (0.07, 0.42) | |

### EEG Power Analysis in Non-Alpha Frequency Bands

Additional post-hoc EEG power analyses were performed in non-alpha frequency bands, including delta (1-4 Hz), theta (4-7 Hz), beta (13-30 Hz), and gamma (30-50 Hz). The pre-processing for analyses in these additional frequency bands was identical to what was performed prior to analysis in the alpha band as described herein. Spectral analysis was performed in the manner described herein. For the attenuation condition, attenuation was calculated in the same manner as alpha as described herein. The topographic region of interest analysis was performed in a similar manner as for alpha power bands described herein, with the same electrodes assigned to the same regions of interest as shown in FIG. 4. Normality and the associated univariate calculations impacted by non-normality in the delta, theta, and gamma bands were analyzed in the same manner as alpha as described herein.

Multivariable models corrected for age and Mini-Mental Status Exam (MMSE) score were generated evaluating possible relationships between whole head EEG across the delta, theta, beta, and gamma frequency bands for three conditions: attenuation, eyes closed and eyes open. This was performed for four outcomes of interest: inattention presence/absence, inattention chronicity, delirium incidence, and delirium severity.

The 12 whole-head models (4 frequency bands×3 states) shown in Table 15 for the inattention status outcome mirror Table 3; these analyses showed no significant effects for inattention status in the multivariate models for these other frequency bands.

TABLE 15
Post-hoc preoperative non-alpha frequency analysis for attenuation
with eyes opening and postoperative attention status.

| | | | Univariate | Multivariate | |
|---|---|---|---|---|---|
| Attentive | Inattentive | Mean Difference | T- Adjusted | Age- and MMSE-adjusted | |
| (N = 31) | (N = 40) | dB (95% CI) or | T-test or | logistic regression with | |
| dB (SD) or | dB (SD) or | HL Location | Wilcoxon | attention status¹ | |
| median | median | Shift | rank sum | OR | Adjusted |
| [Q1, Q3] | [Q1, Q3] | (95% CI) | p value² | (95% CI) | p-value² |

Attenuation (Eyes Closed Minus Eyes Open)

| | | | | | | |
|---|---|---|---|---|---|---|
| Delta | 0.2 | 0.6 | −0.2 | >0.9999 | 0.89 | >0.9999 |
| (1-4 Hz) | [−0.6, 1.4] | [−0.4, 1.0] | (−0.75, 0.61) | | (0.65, 1.23) | |
| Theta | 1.3 | 1.0 | 0.3 | >0.9999 | 0.89 | >0.9999 |
| (4-7 Hz) | [0.5, 3.4] | [0.4, 2.5] | (−0.45, 0.96) | | (0.66, 1.2) | |
| Beta | 1.0 | 0.1 | 0.8 | 0.0448 | 0.6 | 0.1452 |
| (13-30 Hz) | (1.4) | (1.3) | (0.2, 1.5) | | (0.37, 0.97) | |
| Gamma | −0.9 | −0.7 | 0.2 | >0.9999 | 0.89 | >0.9999 |
| (30-50 Hz) | [−1.8, 1.5] | [−1.9, 0.3] | (−0.68, 1.27) | | (0.7, 1.13) | |

Eyes Closed

| | | | | | | |
|---|---|---|---|---|---|---|
| Delta | 7.9 | 7.5 | −0.1 | >0.9999 | 1.13 | 0.9177 |
| (1-4 Hz) | [6.1, 9.2] | [6.4, 9.9] | (−1.31, 1.14) | | (0.89, 1.43) | |
| Theta | 5.5 | 5.2 | −0.4 | >0.9999 | 1.14 | 0.606 |
| (4-7 Hz) | [3.8, 6.5] | [3.5, 8.7] | (−2.2, 1.01) | | (0.95, 1.37) | |
| Beta | 9.8 | 10.0 | −0.1 | >0.9999 | 1.06 | >0.9999 |
| (13-30 Hz) | (2.5) | (3.2) | (−1.5, 1.2) | | (0.88, 1.28) | |
| Gamma | 3.6 | 3.9 | −0.6 | >0.9999 | 1.03 | >0.9999 |
| (30-50 Hz) | [1.0, 6.1] | [1.8, 6.4] | (−2.21, 1.09) | | (0.85, 1.24) | |

Eyes Open

| | | | | | | |
|---|---|---|---|---|---|---|
| Delta | 7.1 | 7.4 | −0.5 | 0.3941 | 1.24 | 0.3195 |
| (1-4 Hz) | [5.7, 8.5] | [6.5, 9.4] | (−1.6, 0.62) | | (0.96, 1.6) | |
| Theta | 3.8 | 4.6 | −1.1 | 0.3567 | 1.29 | 0.1304 |
| (4-7 Hz) | [1.7, 4.9] | [2.8, 6.7] | (−2.5, 0.22) | | (1.02, 1.63) | |
| Beta | 8.8 | 9.8 | −1 | 0.3567 | 1.17 | 0.3195 |
| (13-30 Hz) | (2.6) | (3.0) | (−2.3, 0.4) | | (0.96, 1.42) | |
| Gamma | 3.2 | 4.7 | −1.4 | 0.2856 | 1.15 | 0.3195 |
| (30-50 Hz) | [1.8, 5.8] | [3.7, 6.3] | (−2.65, 0.15) | | (0.92, 1.43) | |

¹EEG numeric variables were summarized with mean (SD). Eyes-closed and eyes-open analyses are included for completeness. Parallel alpha and alpha sub-band data is presented in Table 3.

The 12 whole-head models in Table 16 for the inattention chronicity outcome mirror Table 8. There were two significant differences observed for the inattention chronicity outcome, one for delta band and one for the theta band, in the eyes-open state only. These effects may reflect the known EEG slowing observed in more vulnerable brains, and these findings are consistent with the significantly greater low alpha (7-10 Hz), but not high alpha (10-13 Hz), power observed for chronically inattentive patients in the eyes-open state in Table 8. That said, these are exploratory post-hoc analyses with multiple comparison correction performed within a state (e.g., eyes closed) and outcome (e.g., inattention chronicity), but not across states and outcomes.

TABLE 16
Post-hoc preoperative non-alpha frequency analysis for attenuation
with eyes opening and postoperative inattention chronicity.

| | | | | Multivariate | |
|---|---|---|---|---|---|
| | Newly | Chronically | Univariate | Age- and MMSE- Adjusted | |
| Attentive | Inattentive | Inattentive | ANOVA or | Proportional Odds regression | |
| (N = 31) | (N = 22) | (N = 18) | Kruskal | for greater inattention | |
| dB (SD) | dB (SD) | dB (SD) | Wallis | chronicity¹ | |
| or median | or median | or median | adjusted | OR | Adjusted |
| [Q1, Q3] | [Q1, Q3] | [Q1, Q3] | p value² | (95% CI) | p-value² |

Attenuation (Eyes Closed Minus Eyes Open)

| | | | | | | |
|---|---|---|---|---|---|---|
| Delta | 0.2 | 0.4 | 0.8 | >0.9999 | 0.97 | >0.9999 |
| (1-4 Hz) | [−0.6, 1.4] | [−0.3, 0.8] | [−0.5, 1.6] | | (0.75, 1.27) | |
| Theta | 1.3 | 1.0 | 0.9 | >0.9999 | 0.97 | >0.9999 |
| (4-7 Hz) | [0.5, 3.4] | [0.4, 2.5] | [0.3, 2.5] | | (0.75, 1.25) | |
| Beta | 1.0 | 0.1 | 0.2 | 0.1556 | 0.8 | 0.8684 |
| (13-30 Hz) | (1.4) | (1.1) | (1.5) | | (0.56, 1.14) | |
| Gamma | −0.9 | −0.7 | −0.7 | >0.9999 | 0.94 | >0.9999 |
| (30-50 Hz) | [−1.8, 1.5] | [−1.8, 0.6] | [−2.2, 0.2] | | (0.77, 1.15) | |

Eyes Closed

| | | | | | | |
|---|---|---|---|---|---|---|
| Delta | 7.9 | 7.1 | 8.6 | >0.9999 | 1.23 | 0.1404 |
| (1-4 Hz) | [6.1, 9.2] | [6.4, 9.6] | [6.4, 12.4] | | (1.01, 1.5) | |
| Theta | 5.5 | 4.6 | 5.9 | >0.9999 | 1.17 | 0.1404 |
| (4-7 Hz) | [3.8, 6.5] | [3.3, 8.2] | [3.8, 9.0] | | (1.01, 1.35) | |
| Beta | 9.8 | 10.0 | 9.9 | >0.9999 | 1.06 | 0.9744 |
| (13-30 Hz) | (2.5) | (3.5) | (2.9) | | (0.9, 1.25) | |
| Gamma | 3.6 | 3.9 | 3.9 | >0.9999 | 0.99 | 0.9744 |
| (30-50 Hz) | [1.0, 6.1] | [1.4, 6.5] | [2.0, 5.5] | | (0.85, 1.16) | |

Eyes Open

| | | | | | | |
|---|---|---|---|---|---|---|
| Delta | 7.1 | 7.1 | 7.7 | 0.6518 | 1.3 | 0.0453* |
| (1-4 Hz) | [5.7, 8.5] | [6.1, 8.6] | [6.8, 11.2] | | (1.05, 1.6) | |
| Theta | 3.8 | 3.9 | 4.9 | 0.4964 | 1.27 | 0.0252* |
| (4-7 Hz) | [1.7, 4.9] | [2.7, 5.7] | [3.7, 8.3] | | (1.07, 1.52) | |
| Beta | 8.8 | 9.9 | 9.7 | 0.6518 | 1.11 | 0.4086 |
| (13-30 Hz) | (2.6) | (3.1) | (2.9) | | (0.94, 1.31) | |
| Gamma | 3.2 | 4.9 | 4.7 | 0.5742 | 1.04 | 0.6597 |
| (30-50 Hz) | [1.8, 5.8] | [3.6, 6.4] | [3.9, 5.3] | | (0.87, 1.24) | |

¹EEG numeric variables were summarized with mean (SD). Eyes-closed and eyes-open analyses are included for completeness. Parallel alpha and alpha sub-band data is presented in Table 8.

The 12 whole-head models in Table 17 for the delirium incidence outcome mirrors Table 11, while the 12 whole-head models in Table 18 for delirium severity mirror Table 10; these analyses for delirium incidence and delirium severity also showed no significant effects in the multivariate models for these other frequency bands.

TABLE 17
Post-hoc preoperative non-alpha frequency analysis for attenuation with eyes opening
and postoperative delirium incidence. For the EEG numeric variables, we summarize
with mean (SD). Eyes-closed and eyes-open analyses are included for completeness.
Parallel alpha and alpha sub-band data is presented in Table 11.

| Univariate | | | | | |
|---|---|---|---|---|---|
| No POD | POD | | | Multivariate | |
| (N = 60) | (N = 11) | Mean | Adjusted | Age and MMSE Adjusted | |
| dB mean | dB mean | Difference | T-test or | Logistic Regression delirious | |
| (SD) or | (SD) or | dB (95% CI) or | Wilcoxon | vs non-delirious | |
| median | median | HL Location | rank sum | | Adjusted |
| [Q1, Q3] | [Q1, Q3] | Shift (95% CI) | p value¹ | OR (95% CI) | p-value¹ |

Attenuation (Eyes Closed Minus Eyes Open)

| | | | | | | |
|---|---|---|---|---|---|---|
| Delta | 0.3 | 0.6 | 0.2 | >0.9999 | 0.85 | 0.7914 |
| (1-4 Hz) | [−0.5, 1.1] | [−0.0, 1.5] | (−0.94, 0.96) | | (0.51, 1.43) | |
| Theta | 1.0 | 0.8 | −0.4 | >0.9999 | 0.77 | 0.7914 |
| (4-7 Hz) | [0.5, 2.9] | [−0.1, 2.5] | (−1.39, 1.06) | | (0.48, 1.22) | |
| Beta | 0.5 | 0.6 | −0.2 | >0.9999 | 1.37 | 0.7914 |
| (13-30 Hz) | (1.3) | (1.6) | (−1.1, 0.7) | | (0.79, 2.38) | |
| Gamma | −0.8 | 0.2 | 0.9 | >0.9999 | 1.44 | 0.1528 |
| (30-50 Hz) | [−1.9, 0.5] | [−1.8, 2.4] | (−0.8, 2.66) | | (1.02, 2.04) | |

Eyes Closed

| | | | | | | |
|---|---|---|---|---|---|---|
| Delta | 7.7 | 7.5 | −1 | >0.9999 | 0.88 | >0.9999 |
| (1-4 Hz) | [6.5, 9.7] | [6.3, 8.1] | (−2.78, 0.61) | | (0.66, 1.16) | |
| Theta | 5.4 | 5.1 | −0.5 | >0.9999 | 0.89 | >0.9999 |
| (4-7 Hz) | [3.7, 7.3] | [2.6, 7.5] | (−2.83, 1.6) | | (0.72, 1.1) | |
| Beta | 10.0 | 9.4 | 0.6 | >0.9999 | 0.92 | >0.9999 |
| (13-30 Hz) | (2.8) | (3.4) | (−1.3, 2.5) | | (0.7, 1.2) | |
| Gamma | 3.7 | 3.5 | 0.2 | >0.9999 | 1.03 | >0.9999 |
| (30-50 Hz) | [1.4, 6.2] | [1.6, 7.7] | (−2.17, 2.49) | | (0.81, 1.3) | |

Eyes Open

| | | | | | | |
|---|---|---|---|---|---|---|
| Delta | 7.3 | 7.2 | −0.4 | >0.9999 | 0.91 | >0.9999 |
| (1-4 Hz) | [6.1, 9.4] | [6.1, 7.7] | (−2.36, 0.87) | | (0.68, 1.22) | |
| Theta | 4.0 | 4.5 | −0.1 | >0.9999 | 0.92 | >0.9999 |
| (4-7 Hz) | [2.5, 5.6] | [1.8, 5.7] | (−2.26, 1.58) | | (0.73, 1.17) | |
| Beta | 9.5 | 8.7 | 0.8 | >0.9999 | 0.84 | 0.7800 |
| (13-30 Hz) | (2.8) | (3.3) | (−1.1, 2.6) | | (0.63, 1.12) | |
| Gamma | 4.6 | 3.9 | −0.6 | >0.9999 | 0.82 | 0.7800 |
| (30-50 Hz) | [2.4, 6.3] | [1.9, 5.3] | (−2.38, 1.34) | | (0.61, 1.11) | |

TABLE 18
Post-hoc preoperative non-alpha frequency analysis for attenuation with eyes opening
and postoperative delirium severity. For the EEG numeric variables, we summarize
with mean (SD). Eyes-closed and eyes-open analyses are included for completeness.
Parallel alpha and alpha sub-band data is presented in Table 10.

| | | Multivariate | |
|---|---|---|---|
| | | Age- and MMSE-Adjusted | |
| Univariate | | Proportional Odds Regression | |
| Spearman | | for greater delirium severity | |
| Correlation | Adjusted p- | OR | Adjusted p- |
| (95% CI) | value¹ | (95% CI) | value¹ |

Attenuation (Eyes Closed Minus Eyes Open)

| | | | | | | |
|---|---|---|---|---|---|---|
| Delta (1-4 Hz) | 0.06 | (−0.18, 0.29) | >0.9999 | 0.87 | (0.67, 1.13) | 0.9060 |
| Theta (4-7 Hz) | −0.14 | (−0.36, 0.09) | 0.7700 | 0.85 | (0.66, 1.09) | 0.7896 |
| Beta (13-30 Hz) | −0.16 | (−0.38, 0.08) | 0.7700 | 0.93 | (0.67, 1.3) | >0.9999 |
| Gamma (30-50 Hz) | 0.01 | (−0.23, 0.24) | >0.9999 | 1.04 | (0.86, 1.26) | >0.9999 |

Eyes Closed

| | | | | | | |
|---|---|---|---|---|---|---|
| Delta (1-4 Hz) | 0.01 | (−0.23, 0.24) | >0.9999 | 1.05 | (0.88, 1.25) | >0.9999 |
| Theta (4-7 Hz) | 0.11 | (−0.13, 0.33) | >0.9999 | 1.07 | (0.95, 1.21) | >0.9999 |
| Beta (13-30 Hz) | 0.07 | (−0.17, 0.3) | >0.9999 | 1.09 | (0.93, 1.27) | >0.9999 |
| Gamma (30-50 Hz) | 0.05 | (−0.19, 0.28) | >0.9999 | 1.01 | (0.87, 1.16) | >0.9999 |

Eyes Open

| | | | | | | |
|---|---|---|---|---|---|---|
| Delta (1-4 Hz) | 0.11 | (−0.13, 0.33) | 0.7881 | 1.13 | (0.94, 1.37) | 0.5733 |
| Theta (4-7 Hz) | 0.23 | (−0.01, 0.44) | 0.2264 | 1.18 | (1.01, 1.37) | 0.1444 |
| Beta (13-30 Hz) | 0.13 | (−0.1, 0.36) | 0.7881 | 1.11 | (0.95, 1.29) | 0.5733 |
| Gamma (30-50 Hz) | 0.12 | (−0.12, 0.34) | 0.7881 | 0.97 | (0.82, 1.15) | 0.7426 |

Then, eyes-closed minus eyes-open power attenuation in each topographic region of interest for each condition and each outcome was evaluated. Table 19 shows the 16 models of the relationship between delta, theta, beta, and gamma attenuation in the frontal, central, parietal, and occipital regions of interests and postoperative inattention incidence, which mirrors alpha power findings presented in the left side of Table 12.

TABLE 19
Post-hoc preoperative non-alpha frequency analysis
for attenuation with eyes opening and postoperative
inattention status by region of interest.

| Age- and MMSE-adjusted logistic regression, alpha | | |
|---|---|---|
| attenuation, and attentive vs inattentive status | | |
| OR | Uncorrected | Adjusted¹ |
| (95% CI) | p-value | p-value |

Delta (1-4 Hz)

| | | | | |
|---|---|---|---|---|
| Frontal | 0.94 | (0.75, 1.17) | 0.5803 | >0.9999 |
| Central | 0.82 | (0.57, 1.16) | 0.2590 | >0.9999 |
| Parietal | 0.78 | (0.54, 1.11) | 0.1595 | >0.9999 |
| Occipital | 0.93 | (0.66, 1.3) | 0.6674 | >0.9999 |

Theta (4-7 Hz)

| | | | | |
|---|---|---|---|---|
| Frontal | 0.9 | (0.69, 1.17) | 0.4214 | >0.9999 |
| Central | 0.82 | (0.61, 1.12) | 0.2191 | >0.9999 |
| Parietal | 0.85 | (0.65, 1.12) | 0.2547 | >0.9999 |
| Occipital | 1.01 | (0.8, 1.27) | 0.9647 | >0.9999 |

Beta (13-30 Hz)

| | | | | |
|---|---|---|---|---|
| Frontal | 0.51 | (0.3, 0.88) | 0.0161 | 0.2254 |
| Central | 0.51 | (0.29, 0.9) | 0.0201 | 0.2613 |
| Parietal | 0.5 | (0.29, 0.84) | 0.0092 | 0.1472 |
| Occipital | 0.61 | (0.42, 0.9) | 0.0127 | 0.1905 |

Gamma (30-50 Hz)

| | | | | |
|---|---|---|---|---|
| Frontal | 0.84 | (0.66, 1.08) | 0.1735 | >0.9999 |
| Central | 0.82 | (0.63, 1.07) | 0.1474 | >0.9999 |
| Parietal | 0.84 | (0.66, 1.08) | 0.1746 | >0.9999 |
| Occipital | 0.88 | (0.68, 1.12) | 0.2983 | >0.9999 |

Table 20 shows the 16 models of the relationship between delta, theta, beta, and gamma attenuation in the frontal, central, parietal and occipital regions of interests and postoperative inattention chronicity, which mirrors alpha power findings presented in the right side of Table 12.

TABLE 20
Post-hoc preoperative non-alpha frequency analysis
for attenuation with eyes opening and postoperative
inattention chronicity by region of interest.

| Age- and MMSE-adjusted proportional odds regression for | | |
|---|---|---|
| alpha attenuation and greater inattention chronicity | | |
| OR | Uncorrected | Adjusted¹ |
| (95% CI) | p-value | p-value |

Delta (1-4 Hz)

| | | | | |
|---|---|---|---|---|
| Frontal | 0.95 | (0.78, 1.16) | 0.6195 | >0.9999 |
| Central | 0.92 | (0.69, 1.22) | 0.5565 | >0.9999 |
| Parietal | 0.88 | (0.66, 1.16) | 0.3553 | >0.9999 |
| Occipital | 0.99 | (0.74, 1.33) | 0.9716 | >0.9999 |

Theta (4-7 Hz)

| | | | | |
|---|---|---|---|---|
| Frontal | 0.94 | (0.75, 1.16) | 0.5536 | >0.9999 |
| Central | 0.92 | (0.71, 1.19) | 0.5281 | >0.9999 |
| Parietal | 0.91 | (0.72, 1.15) | 0.4175 | >0.9999 |
| Occipital | 1 | (0.82, 1.23) | 0.9857 | >0.9999 |

Beta (13-30 Hz)

| | | | | |
|---|---|---|---|---|
| Frontal | 0.7 | (0.48, 1.01) | 0.0560 | 0.8960 |
| Central | 0.77 | (0.53, 1.12) | 0.1641 | >0.9999 |
| Parietal | 0.77 | (0.55, 1.07) | 0.1208 | >0.9999 |
| Occipital | 0.81 | (0.62, 1.06) | 0.1271 | >0.9999 |

Gamma (30-50 Hz)

| | | | | |
|---|---|---|---|---|
| Frontal | 0.88 | (0.71, 1.08) | 0.2062 | >0.9999 |
| Central | 0.89 | (0.72, 1.1) | 0.2919 | >0.9999 |
| Parietal | 0.93 | (0.76, 1.12) | 0.4390 | >0.9999 |
| Occipital | 0.95 | (0.78, 1.17) | 0.6508 | >0.9999 |

Table 21 shows the 16 models of the relationship between attenuation in the four non-alpha frequency bands in the four topographic regions of interests and postoperative delirium incidence, which mirrors alpha power findings presented in Table 22.

TABLE 21
Post-hoc preoperative non-alpha frequency analysis
for attenuation with eyes opening and postoperative
delirium incidence by region of interest.

| Age- and MMSE-Adjusted Logistic | | |
|---|---|---|
| Regression for delirious vs non-delirious | | |
| OR | Uncorrected | Adjusted¹ |
| (95% CI) | p-value | p-value |

Delta (1-4 Hz)

| | | | | |
|---|---|---|---|---|
| Frontal | 1 | (0.72, 1.38) | 0.9813 | >0.9999 |
| Central | 0.68 | (0.37, 1.25) | 0.2145 | >0.9999 |
| Parietal | 0.73 | (0.42, 1.25) | 0.2515 | >0.9999 |
| Occipital | 0.88 | (0.53, 1.48) | 0.6402 | >0.9999 |

Theta (4-7 Hz)

| | | | | |
|---|---|---|---|---|
| Frontal | 0.83 | (0.57, 1.21) | 0.3322 | >0.9999 |
| Central | 0.65 | (0.39, 1.09) | 0.1042 | >0.9999 |
| Parietal | 0.75 | (0.49, 1.15) | 0.1810 | >0.9999 |
| Occipital | 0.86 | (0.58, 1.27) | 0.4454 | >0.9999 |

Beta (13-30 Hz)

| | | | | |
|---|---|---|---|---|
| Frontal | 1.11 | (0.64, 1.93) | 0.7127 | >0.9999 |
| Central | 1.05 | (0.58, 1.9) | 0.8682 | >0.9999 |
| Parietal | 1.09 | (0.66, 1.79) | 0.7473 | >0.9999 |
| Occipital | 1.33 | (0.86, 2.07) | 0.1997 | >0.9999 |

Gamma (30-50 Hz)

| | | | | |
|---|---|---|---|---|
| Frontal | 1.33 | (0.95, 1.87) | 0.1004 | >0.9999 |
| Central | 1.28 | (0.94, 1.72) | 0.1123 | >0.9999 |
| Parietal | 1.26 | (0.96, 1.65) | 0.0981 | >0.9999 |
| Occipital | 1.6 | (1.09, 2.34) | 0.0153 | 0.2448 |

TABLE 22
Multivariate model of pre-operative alpha attenuation
by spatial region and post-operative delirium status.

| | Age- and MMSE-Adjusted Logistic | | |
|---|---|---|---|
| | Regression for delirious vs non-delirious | | |
| | OR | Uncorrected | Adjusted |
| | (95% CI) | p-value | p-value¹ |
| Frontal | 0.93 (0.72, 1.20) | 0.5661 | >0.999 |
| Central | 0.90 (0.65, 1.24) | 0.5295 | >0.999 |
| Parietal | 0.97 (0.75, 1.26) | 0.8191 | >0.999 |
| Occipital | 0.99 (0.79, 1.24) | 0.9297 | >0.999 |

Table 23 shows the 16 models of the relationship between attenuation in the four non-alpha frequency bands in the four topographic regions of interests and postoperative delirium severity, which mirrors alpha power findings presented in Table 13. In sum, more than 100 post-hoc exploratory models are presented here with non-alpha frequency analyses.

TABLE 23
Post-hoc preoperative non-alpha frequency analysis for
attenuation with eyes opening and delirium severity.

| Age- and MMSE- Adjusted Proportional Odds | | |
|---|---|---|
| Regression for greater delirium severity | | |
| OR | Uncorrected | Adjusted¹ |
| (95% CI) | p-value | p-value |

Delta (1-4 Hz)

| | | | | |
|---|---|---|---|---|
| Frontal | 0.93 | (0.78, 1.11) | 0.4308 | >0.9999 |
| Central | 0.83 | (0.63, 1.09) | 0.1889 | >0.9999 |
| Parietal | 0.78 | (0.59, 1.03) | 0.0790 | >0.9999 |
| Occipital | 0.91 | (0.69, 1.2) | 0.4915 | >0.9999 |

Theta (4-7 Hz)

| | | | | |
|---|---|---|---|---|
| Frontal | 0.87 | (0.71, 1.08) | 0.1995 | >0.9999 |
| Central | 0.84 | (0.65, 1.08) | 0.1769 | >0.9999 |
| Parietal | 0.85 | (0.67, 1.06) | 0.1493 | >0.9999 |
| Occipital | 0.98 | (0.81, 1.19) | 0.8669 | >0.9999 |

Beta (13-30 Hz)

| | | | | |
|---|---|---|---|---|
| Frontal | 0.8 | (0.57, 1.12) | 0.1992 | >0.9999 |
| Central | 0.89 | (0.63, 1.25) | 0.5007 | >0.9999 |
| Parietal | 0.93 | (0.69, 1.26) | 0.6325 | >0.9999 |
| Occipital | 1 | (0.78, 1.28) | 0.9770 | >0.9999 |

Gamma (30-50 Hz)

| | | | | |
|---|---|---|---|---|
| Frontal | 1 | (0.83, 1.2) | 0.9914 | >0.9999 |
| Central | 1.03 | (0.86, 1.24) | 0.7561 | >0.9999 |
| Parietal | 1.02 | (0.86, 1.22) | 0.7939 | >0.9999 |
| Occipital | 1.08 | (0.9, 1.3) | 0.4143 | >0.9999 |

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A system for detecting neurocognitive weakness in a subject comprising:
one or more electrodes operable to measure a first set of one or more EEG signals of the subject and a second set of one or more EEG signals of the subject; and
at least one processor configured to:
receive the first set of one or more EEG signals and the second set of one or more EEG signals;
determine a first alpha power for the first set of one or more EEG signals and a second alpha power for the second set of one or more EEG signals;
determine a difference between the first alpha power and the second alpha power; and
generate an alpha reactivity defined by the difference,
wherein the first set of one or more EEG signals is measured when the subject is in an eyes closed state, and
wherein the second set of one or more EEG signals is measured when the subject is in an eyes open state.

2. The system of claim 1, wherein the at least one processor is further configured to compare the alpha reactivity to a threshold or degree of alpha reactivity.

3. The system of claim 2, wherein the at least one processor is further configured to determine, based on the comparison of the alpha reactivity to the threshold or the degree of alpha reactivity, an attentiveness of the subject.

4. The system of claim 3, wherein the attentiveness of the subject comprises one of attentive, likely to become inattentive, moderately inattentive, and chronically inattentive.

5. The system of claim 1, wherein the one or more electrodes comprise at least two frontal electrodes.

6. The system of claim 5, wherein the at least two frontal electrodes are operable to contact a forehead of the subject.

7. The system of claim 1, wherein the one or more electrodes further comprises at least one occipital electrode and at least one frontal electrode.

8. The system of claim 7, wherein the at least one occipital electrode is operable to contact a scalp of the subject over an occipital lobe of the subject.

9. The system of claim 1, wherein the at least one processor is further configured to separate the first alpha power and the second alpha power into one or more sub-bands, wherein the one or more sub-bands include a low-frequency sub-band and a high-frequency sub-band.

10. The system of claim 9, wherein the at least one processor is further configured to:
determine a difference between the first alpha power and the second alpha power in the one or more sub-bands;
generate one or more sub-band alpha reactivities for each of the one or more sub-bands; and
determine one or more conditions of the subject based on the one or more sub-band alpha reactivities.

11. The system of claim 1, wherein the at least one processor is further operable to determine a treatment for the subject based on the alpha reactivity.

12. The system of claim 1, further comprising a support structure configured to be worn by the subject, wherein the one or more electrodes are coupled to the support structure.

13. A method for detecting and treating neurocognitive weakness in a subject, the method comprising:
measuring, via one or more electrodes in contact with the subject, a first set of one or more EEG signals of the subject and a second set of one or more EEG signals of the subject;
sending the first set of one or more EEG signals and the second set of one or more EEG signals to at least one processor;
determining, via the at least one processor, a first alpha power for the first set of one or more EEG signals and a second alpha power for the second set of one or more EEG signals;
determining a difference between the first alpha power and the second alpha power;
generating an alpha reactivity defined by the difference;
comparing the alpha reactivity to one or more alpha reactivity thresholds or degrees;
determining a treatment based on the comparison of the alpha reactivity to the one or more alpha reactivity thresholds or degrees; and
administering the treatment to the subject,
wherein the first set of one or more EEG signals is measured when the subject is in an eyes closed state, and
wherein the second set of one or more EEG signals is measured when the subject is in an eyes open state.

14. The method of claim 13, further comprising determining, based on the comparison of the alpha reactivity to the one or more alpha reactivity thresholds or degrees, a robustness of an attentional or cognitive control system of the subject.

15. The method of claim 14, wherein the robustness of the attentional or cognitive control system comprises one of attentive, likely to become inattentive, moderately inattentive, and chronically inattentive.

16. The method of claim 13, further comprising separating the first alpha power and the second alpha power into one or more sub-bands.

17. The method of claim 16, further comprising:
determining a difference between the first alpha power and the second alpha power for each of the one or more sub-bands;
generating one or more alpha sub-band reactivities for each of the one or more sub-bands; and
determining one or more conditions of the subject based on the one or more alpha sub-band reactivities.

18. The method of claim 17, wherein the one or more conditions include one or more of inattentiveness, post-operative delirium, MCI, dementia, depression, anxiety, ADHD, and other neurological conditions.

19. The method of claim 16, wherein the one or more sub-bands include a lower frequency sub-band and a high frequency sub-band.

20. The method of claim 13, wherein the treatment includes one or more of neurofeedback or biofeedback therapy, family counseling, administration of medications, and/or adjustment of anesthesia-inducing procedures and/or drugs.
